(19) United States
(12) Patent Application Publication
(10) Pub. No.: US 2011/0313167 A1
(43) Pub. Date: Dec. 22, 2011

(54) Substituted Heterocycles as Therapeutic agents for treating cancer

(75) Inventor: Alexander Doemling, Pittsburgh, PA (US)

(73) Assignee: University of Pittsburgh - Of the Commonwealth System of Higher Education

(21) Appl. No.: 13/067,700

(22) Filed: Jun. 21, 2011

Related U.S. Application Data
(60) Provisional application No. 61/357,365, filed on Jun. 22, 2010.

(51) Int. Cl. C07D 401/12

(52) U.S. Cl. 546/274.7

(57) ABSTRACT

MDM2 and MDM4 proteins prevent apoptosis of cancer cells by negatively regulating the transcription factor p53. Compounds according to Formula I

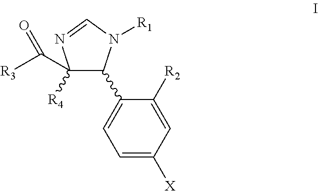

are selective antagonists of MDM2 and MDM4 proteins, disrupting the p53/MDM2 and p53/MDM4 complex. These compounds therefore are candidate therapeutics for treating cancer as well as other cell proliferative disease states.

### CROSS-REFERENCE TO RELATED PATENT APPLICATIONS

This application claims priority to U.S. provisional application Ser. No. 61/357,365, filed Jun. 22, 2010, the entire contents of which are incorporated herein by reference.

### BACKGROUND OF THE INVENTION

The p53 protein is a tumor suppressor protein. Disruption of the genetic machinery encoding this protein or a disruption in the normal physiological function of p53 has been observed to accompany about 50% of all cancers. The p53 protein serves as a checkpoint during cell division and this protein prevents cancers by activating DNA repair proteins, by inducing cell growth arrest, or by initiating apoptosis. The, p53 protein is also implicated to play a role in the development of tumors that become resistant to treatment. It therefore follows that the p53 proteins plays a key role in controlling the progression of cancer.

The ability of p53 to initiate programmed cell death is most often repressed in cancer. Of the variety of biological molecules that are capable of inactivating p53, the oncoprotein MDM2 is believed to be the main negative regulator. Recently, another p53-binding protein, MDM4 (MDMX), has gained increasing attention as an equally important negative regulator of p53. In particular, a consensus exists that effective activation of p53-induced apoptosis must be based on a dual-action approach, involving both MDM2 and MDM4 antagonism. Thus, dual-action p53/MDM2/MDM4 antagonists can be used to treat cancer, and so might represent an important class of anti-cancer drugs. See Toledo & Wahl, Nat. Rev. Cancer 6: 909-23 (2006). In the present context, “hMDM2” and “Hdm2” are used interchangeably.

There are no known, small-molecule MDM4 inhibitors, and no small-molecule therapeutic been identified that is capable of dual-action MDM2/MDM4 antagonism. Furthermore, in the absence of structural data, such as a high resolution structure of p53 bound to the MDM4 protein, the development of molecules capable of inhibiting or disrupting the p53/MDM4 interactions is challenging.

### SUMMARY OF THE INVENTION

In accordance with one aspect of the invention, a compound belonging to the imidazoline class is provided that antagonizes MDM2/p53 or MDM4/p53 complex. Illustrative of such a compound is one that conforms to Formula I,

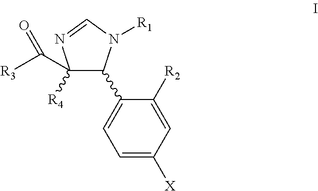

For Formula I compounds, R₁ is selected from the group consisting of phenyl, alkyl, aryl, cycloalkyl, cycloalkylalkylene, arylalkylene, heterocycle, and heterocycloalkyl. When R₁ is phenyl or arylalkylene, R₁ is substituted with one or more substituents selected from the group consisting of (C₁-C₈)alkyl, (C₁-C₈)haloalkyl, —Cl, —Br, —I, —F —NO₂, and (C₁-C₆)hydroxyalkyl.

Substituent X is selected from the group consisting of Cl, F, Br and I. R₂ is selected from the group consisting of —OH and NR^{a}R^{b}, with R^{a} and R^{b} each independently being selected from the group consisting of hydrogen, (C₁-C₈)alkyl, aryl, heteroaryl, heterocycloalkyl, and (C₁-C₆)hydroxyalkyl group.

R₃ is selected from the group consisting of alkoxy, and —NHR^{c} group. When R₃ is a —NHR^{c} group, R^{c} is selected from the group consisting of hydrogen, cycloalkylakylene, alkoxy-(C₁-C₈)alkylene, (C₃-C₈)heterocycloalkyl-(C₁-C₈)alkylene, (C₃-C₈)heteroaryl-(C₁-C₆)alkylene, amino-(C₁-C₈)alkylene, hydroxyalkylene, and (W)—(CH₂)ₘ—O—(CH₂)ₙ—. The group W is selected from the group consisting of —OH, and NR^{a}R^{b}, with m and n each independently being an integer in the range from 1 to 8 inclusive.

Substituent R₄ is selected from the group consisting of aryl, phenyl, benzyl, heteroaryl, heteroaryl-(C₁-C₈)alkylene, and aryl-(C₁-C₈)alkylene, wherein when R₄ is phenyl or benzyl, R₄ is substituted with one or more substituents selected from the group consisting of (C₁-C₈)alkyl, (C₁-C₈)haloalkyl, —Cl, —Br, —I, —F —NO₂, and (C₁-C₆)hydroxyalkyl.

In one embodiment, compounds of this invention are 2-hydroxy-4-halosubstituted imidazolines, such as a 2-hydroxy-4-chlorosubstituted imidazoline. Exemplary compounds according to Formula I are illustrated in Table 1.

The present invention also provides a pharmaceutical composition comprising a therapeutically effective amount of at least one compound according to Formula I,

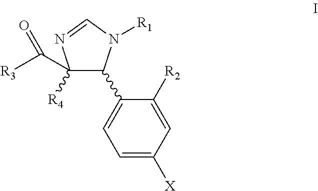

where substituent groups R₁, R₂, R₃, R₄, X, W R^{a}R^{b} and R^{c} are as defined above and subscripts m and n are integers in the range from 1 to 8 inclusive.

### BRIEF DESCRIPTION OF THE DRAWINGS

FIG. 1 shows binding of PB-14 to bind Hdm2 protein. (A). HSQC¹H-¹⁵N HSQC spectra of Hdm2 (1-125) titrated with increasing amount of PB14. Red spectrum is a reference of apo-Hdm2, green spectrum corresponds to approximately 40% saturation of Hdm2 with PB14; the spectrum shows slow chemical exchange that is typical for strong interactions with submicromolar K_{d}s. Blue spectrum corresponds to Hdm2 fully saturated with PB14; (B): perturbation plot of PB14; (C): AIDA-NMR experiment: the concentration of the p53 released from the Hdm2/p53 complex by the antagonist is proportional to the height of the FIN indole peak of W23(p53). The bottom spectrum shows the downfield shifted NMR signals of 20 μM Hdm2/p53 complex, the middle one shows approximately 70% of dissociation of the complex upon addition of PB14 to the complex in 1:1 molar ratio, the upper spectrum shows signals of the free p53; and (D): binding of PB14 by the fluorescent polarization assay.

FIG. 2 illustrates data from NMR-based screening of certain p53-hMDM2 antagonist identified from in silico docking studies. (A): The HSQC perturbation spectra of Hdm2. The spectrum of free Hdm2 (red), and Hdm2 plus syn-PB2 (blue). The final ratio of Hdm2 to syn-PB2 was 1:5; (B): anti-PB2, the spectrum of free Hdm2 (red), and Hdm2 plus anti-PB2 (blue). The final ratio of Hdm2 to anti-PB2 was 1:2; (C): Contact surfaces of Hdm2 for the ligands syn-PB2, PB2 (diastereomeric mixture), PB-11, an PB14. Residues which show significant induced NMR chemical shifts upon complexation with compounds are highlighted in orange and red for observed vectorial shifts of 0.09-0.15 and greater than 0.15 ppm, respectively. The residues of PB 14, that show the slow chemical exchange has been highlighted in dark red.

FIG. 3 illustrates 1D AIDA-NMR data showing disruption of p53-hMDM2 complex in the presence of certain exemplary PB-compounds. For each panel the upper trace corresponds to a spectrum of p53 (residues 1-321). The three peaks in the upper trace correspond to tryptophans of p53, namely, Trp91, Trp23 and Trp53. The middle trace corresponds to a spectrum of the complex of p53 (res. 1-321)+Hdm2 (res. 1-125). Tryptophans 53 and 91 are not sensitive to the binding to Hdm2. Trp23 however is in the binding site and therefore the spectral peak for this residue disappears on binding to Hdm2. The lower trace in each panel corresponds to spectrum obtained in the presence of the following PB-compounds. The dissociation of the p53-hMDM2 complex releases p53 as seen by the reappearance of the spectral peak for tryptophan Trp23. (a): anti-PB2. (b): syn-PB2, (c): PB3, (d): PBS, (e): PB10, (f): PB11.

FIG. 4 illustrates a plot of pK_{D} values (defined as the negative base₁₀ logarithm of the K_{D} value expressed in molar units) vs. molecular weight (MW) for the compounds in Table 3. The dashed line shows the plot expected for the best leads of p53/Hdm2 antagonists. The known p53/Hdm2 antagonists Nutlin-3 and MI-219 are included for reference.

### DETAILED DESCRIPTION

The present invention provides candidate small-molecule therapeutics that are potent dual antagonists of p53/MDM2/MDM4 interactions. In particular, the inventive compounds are shown by Formula I:

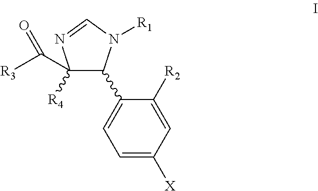

Because compounds of the present invention have asymmetric centers they may occur, except when specifically noted, as mixtures of enantiomers, diastereoisomers or in optically pure form, such as individual enantiomers, or diastereomers, with all isomeric forms being contemplated by the present invention. Compounds of the present invention embrace all conformational isomers, including, for example, cis- and trans conformations.

In one embodiment, for compounds according to Formula I, R₁ is an alkyl group, an aryl group, a cycloalkyl group, a (C₃-C₈)cycloalkyl-(C₁-C₈)alkylene group, an (C₃-C₈)aryl-(C₁-C₈)alkylene group, a heterocycle group, or a heterocycloalkyl group. In one embodiment, R₁ phenyl. When R₁ is a phenyl group or an aryl-(C₁-C₈)alkylene group, R₁ is substituted with one or more substituents selected from the group consisting of (C₁-C₈)alkyl, (C₁-C₈)haloalkyl, —Cl, —Br, —I, —F —NO₂, and (C₁-C₆)hydroxyalkyl.

The present inventor found that for Formula I compounds the presence of a polar group capable of hydrogen bonding interactions at R₂ enhanced binding interactions of the inventive compounds with MDM2 and MDM4 proteins. In one embodiment therefore, the present invention provides Formula I compounds in which R₂ is a hydroxyl group, or an amino group such as a NR^{a}R^{b} group. In this context, the term “amine or amino” refers to —NR^{a}R^{b} group wherein R^{a} and R^{b} each independently refer to a hydrogen, (C₁-C₈)alkyl, aryl, heteroaryl, heterocycloalkyl, and (C₁-C₆)hydroxyalkyl group. In one embodiment of the invention, the —NR^{a}R^{b} group is mono-substituted.

For the inventive compounds, substituent X is a halogen, such as a chlorine, fluorine, bromine or iodine atom. In some embodiments, X is a (C₁-C₈)alkyl, (Cᵣ C₈)haloalkyl, (C₁-C₈)hydroxyalkyl, —OR′, a nitrile (—CN), or an —NR^{a}R^{b} group. In one embodiment, therefore, the inventive compounds are 2-hydroxy-4-cholorophenyl substituted imidazoline derivatives. Binding of the 2-hydroxy-4-cholorophenyl substituted imidazoline derivatives to MDM protein is enhanced because of strong hydrogen bonding interaction between the hydroxyl group and a carbonyl of an active site leucine (Leu54). The present invention also encompasses Formula I compounds where X is halogen and R₂ is a hydrogen.

For compounds of Formula I, substituent R₃ at the C-4 position of the imidazoline ring is an alkoxy group, or an —NHR^{c} group. When R₃ is —NHR^{c}, R^{c} is selected from the group consisting of hydrogen, (C₃-C₈)cycloalkyl-(C₁-C₈)alkylene group, an alkoxy-(C₁-C₈)alkylene group, a (C₃-C₈)heterocycloalkyl-(C₁-C₈)alkylene group, a (C₃-C₈)heteroaryl-(C₁-C₆)alkylene group, an amino-(C₁-C₈)alkylene group, a hydroxyalkylene group or a group according to the formula (W)—(CH₂)ₘ—O—(CH₂)ₙ—. When R^{c} is (W)—(CH₂)ₘ—O—(CH₂)ₙ—, W is either a hydroxyl group, or an —NR^{a}R^{b} group, where R^{a} and R^{b} are as defined above. Subscripts m and n are each independently integers in the range from 1 to 8 inclusive.

For compounds in accordance with the present invention, substituent R₄ is an aryl group, such as a phenyl group, or a benzyl group, a (C₃-C₈)heteroaryl group, a (C₃-C₈)heteroaryl-(C₁-C₈)alkylene group, and an (C₃-C₈)aryl-(C₁-C₈)alkylene group. In embodiments where R₄ is a phenyl group or a benzyl group, R₄ is substituted with one or more substituents selected from the group consisting of (C₁-C₈)alkyl, (C₁-C₈)haloalkyl, —Cl, —Br, —I, —F —NO₂, and (C₁-C₆)hydroxyalkyl.

In the context of the inventive compounds, therefore, the term “alkyl” refers to a straight or branched chain, saturated hydrocarbon having the indicated number of carbon atoms. For example, (C₁-C₈)alkyl is meant to include but is not limited to methyl, ethyl, propyl, isopropyl, butyl, sec-butyl, tert-butyl, pentyl, isopentyl, neopentyl, hexyl, isohexyl, and neohexyl, etc. An alkyl group can be unsubstituted or optionally substituted with one or more substituents as described herein below.

The terms “hydroxyalkyl,” or “hydroxyalkylene” refers to an alkyl group having the indicated number of carbon atoms wherein one or more of the alkyl group's hydrogen atoms is replaced with an —OH group. Examples of hydroxyalkylene groups include but are not limited to —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, —CH₂CH(OH)CH₂OH and branched versions thereof.

The terms “aminoalkyl” or “aminoalkylene” refer to an alkyl group having the indicated number of carbon atoms wherein one or more of the alkyl group's hydrogen atoms is replaced with an —NH₂ group. Examples of aminoalkylene groups include but are not limited to —CH₂ NH₂, —CH₂CH₂ NH₂, —CH₂CH₂CH₂ NH₂, —CH₂CH₂CH₂CH₂ NH₂, —CH₂CH₂CH₂CH₂CH₂ NH₂, —CH₂CH₂CH₂CH₂CH₂CH₂ NH₂, —CH₂CH(NH₂)CH₂ NH₂ and branched versions thereof.

The terms “haloalkyl” or “haloalkylene” refer to an alkyl group having the indicated number of carbon atoms wherein one or more of the alkyl group's hydrogen atoms is replaced with a halogen group (X), where X can be selected from the group consisting of Cl, Br, I and F. Also included within the class of haloalkylene are alkyl groups in which two or more hydrogen atoms are replaced with different halogen atoms. Examples of haloalkylene groups include but are not limited to —CH₂X, —CH₂CH₂X, —CH₂CH₂CH₂X, —CH₂CH₂CH₂CH₂X, —CH₂CH₂CH₂CH₂CH₂X, —CH₂CH₂CH₂CH₂CH₂CH₂X, —CH₂CH(Cl)CH₂Br and branched versions thereof.

The term “aryl” refers to a 3- to 10-membered aromatic hydrocarbon ring system. Examples of an aryl group include phenyl, naphthyl, pyrenyl, and anthracyl. An aryl group can be unsubstituted or optionally substituted with one or more substituents as described herein below.

The term “cycloalkyl” refers to monocyclic, bicyclic, tricyclic, or polycyclic, 3- to 14-membered ring systems, which are either saturated, unsaturated or aromatic. Representative examples of cycloalkyl include but are not limited to cycloethyl, cyclopropyl, cycloisopropyl, cyclobutyl, cyclopentyl, cyclohexyl, cyclopropene, cyclobutene, cyclopentene, cyclohexene, phenyl, naphthyl, anthracyl, benzofuranyl, and benzothiophenyl. A cycloalkyl group can be unsubstituted or optionally substituted with one or more substituents.

The term “cycloalkylalkylene” refers to C₁-C₈ alkylene group in which at least one hydrogen atom of a C₁-C₈ alkylene chain is replaced by an cycloalkyl atom, which may be optionally substituted with one or more substituents. Examples of cycloalkylalkylene groups include but are not limited to methylenecyclopropyl, ethylenecyclopropyl, and butylenecyclopropyl groups.

The term “arylalkylene” denotes a C₁-C₈ alkylene group in which at least one hydrogen atom of the C₁-C₈ alkyl chain is replaced by an aryl atom, which optionally can be substituted with one or more substituents as described below. Examples of this group include but are not limited to methylenephenyl or benzyl, ethylenenaphthyl, propylenephenyl, and butylenephenyl groups.

The term “heteroaryl” denotes a polycyclic aromatic heterocyclic ring system ring of 5 to 18 members, having at least one heteroatom selected from nitrogen, oxygen and sulfur, and containing at least 1 carbon atom, including bicyclic, and tricyclic ring systems.

The terms “heterocycle” and “heterocycloalkyl” refer to bicyclic, tricyclic, or polycyclic systems, which are either unsaturated or aromatic and which contains from 1 to 4 heteroatoms, independently selected from nitrogen, oxygen and sulfur, wherein the nitrogen and sulfur heteroatoms are optionally oxidized and the nitrogen heteroatom optionally quaternized, including bicyclic, and tricyclic ring systems. The bicyclic and tricyclic ring systems may encompass a heterocycle or heteroaryl fused to a benzene ring. The heterocycle may be attached via any heteroatom or carbon atom. Heterocycles include heteroaryls. Representative examples of heterocycles include but are not limited to morpholino, benzoxazolyl, benzisoxazolyl, benzthiazolyl, benzimidazolyl, morisoindolyl, indazolyl, benzodiazolyl, benzotriazolyl, benzoxazolyl, benzisoxazolyl, purinyl, indolyl, isoquinolinyl, quinolinyl and quinazolinyl. A heterocycle group can be unsubstituted or optionally substituted with one or more substituents

In one embodiment, the substituent —CO₂H may be replaced with bioisosteric replacements such as:

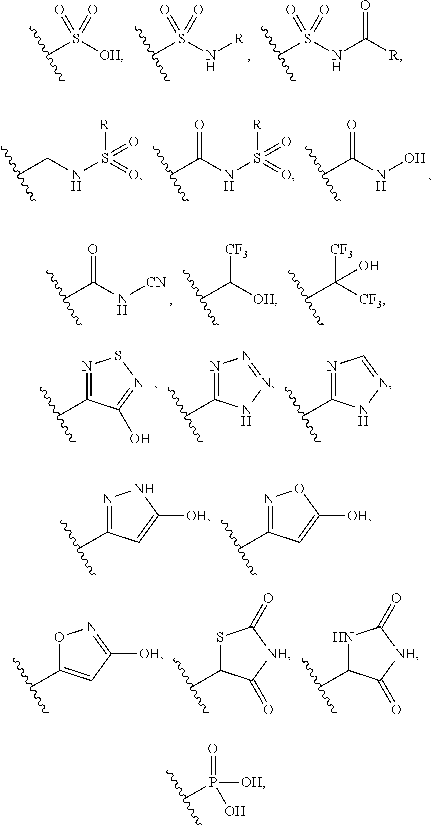

and the like. For example, see THE PRACTICE OF MEDICINAL CHEMISTRY (Academic Press: New York, 1996), at page 203.

The present invention further relates to the discovery of a novel methodology for identifying molecular scaffolds that could selectively disrupt the interaction between the tumor suppressor p53 protein and its negative regulators MDM2 and MDM4. Briefly, this technique utilizes data from high resolution structure studies of p53-MDM2 interaction to identify key amino acids side chains groups (anchor groups) that are directly involved in protein-protein interaction. These anchor groups then are used as a starting point to generate, by means of REACTOR software, a virtual library of compounds comprising all possible stereoisomers. See Pirok, G., et al., J. Chem. Inf. Model. 46: 563-68 (2006).

The p53-hMDM2 complex relies on steric complimentarity between the hMDM2 cleft and the hydrophobic surface of p53. Three residues on the hydrophobic surface of p53, namely, Phe19, Trp23 and Leu26 were identified by X-ray analysis to be critical for complex formation. Of these, Trp23 was chosen as the anchor group because it is buried deep within the hMDM2 cleft and has extensive network of van der Waal contacts to amino acid groups within hMDM2's cleft. Additionally, X-ray studies indicate that Trp23 is within hydrogen bonding distance to the carbonyl group of Leu54 in hMDM2. Accordingly, various aliphatic, 4-membered or 5-membered cyclic or heterocyclic ring systems, or aromatic moieties were chosen as anchors for generating leads using MCR chemistry. Illustrative molecular scaffolds that are suitable as anchors for the in silico synthesis of compounds according to the present invention include, without limitation: imidazoles; imidazolines; thiazoles; indoles; thioxospiro(imidazolidine-4,3′-indolin)-2-one; 4,5,dihydro-1,2,3,5,9b-pentaazacyclopenta[a]naphthalene; and naphthalene.

The virtual compound libraries incorporating the anchor side chain were docked into a rigid model of the p53 binding site in the hMDM2 receptor using the software Moloc. Assuming that the anchor residue predefines the orientation of a compound within hMDM2's binding pocket and to avoid nonproductive docking, the present inventor forced the anchor part of the virtual compounds to overlap the region occupied by Trp23 in p53-hMDM2 complex.

Based on the ranking score for virtual compounds using in silico docking studies, several lead compounds having diverse molecular scaffolds were rapidly identified. The highest ranking compounds were synthesized using multicomponent reaction chemistry (MCR) and screened for activity. Scheme 1 illustrates the backbone structures of chemical scaffolds that were identified by in silico docking studies to be tight binders of hMDM2. These compound, according to the present invention are candidate therapeutics for disrupting the p53-hMDM2 complex in vivo and thus represent a novel class of ntineoplastic agents.

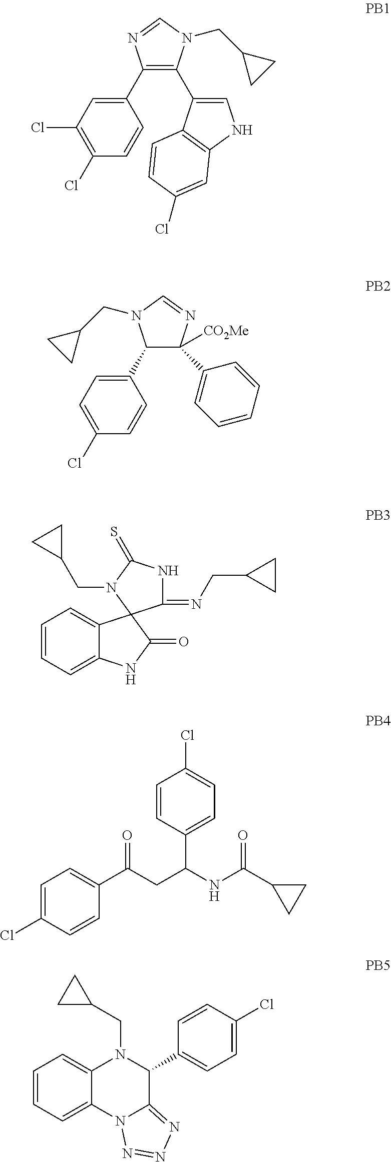

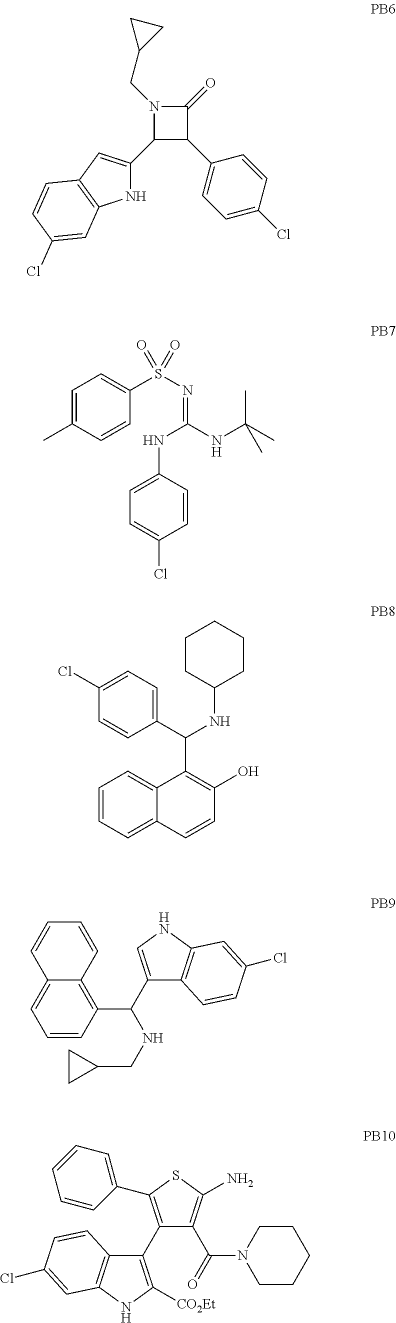

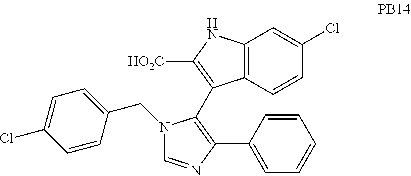

The present invention encompasses, for example, compounds having an imidazole or imidazoline core. Inventive compounds belonging to the imidazoline class are represented by Formula I and were synthesized using the following, known protocols.

Thus, in one aspect, the inventive compounds were synthesized using a 3-component reaction mixture using an aldehyde, an amine and an isocyanide, followed by purification of the crudecompounds using silica gel column chromatography. See Bon, R. S., et al. Novel multicomponent reaction for the combinatorial synthesis of 2-imidazolines. Org. Lett. 5, 3759-3762 (2003). This synthetic methodology was used to prepare several imidazoline derivatives. Illustrative of compounds of Formula I are those shown in Table 1

TABLE 1

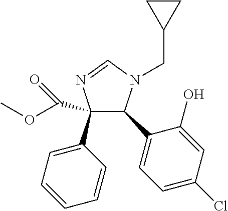

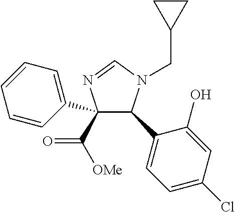

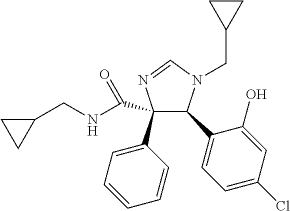

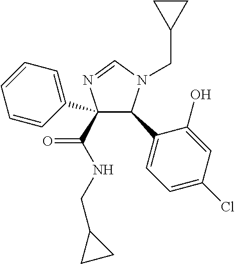

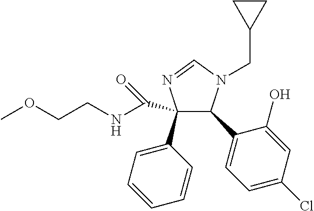

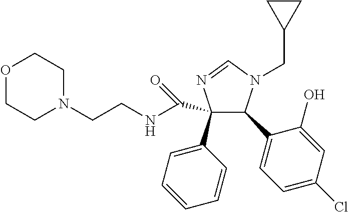

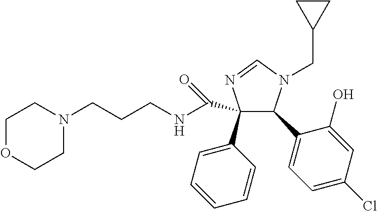

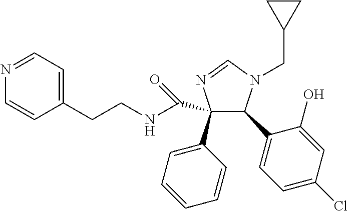

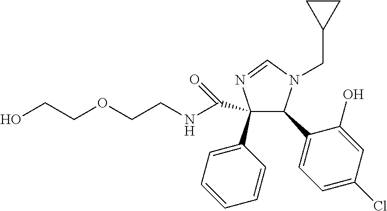

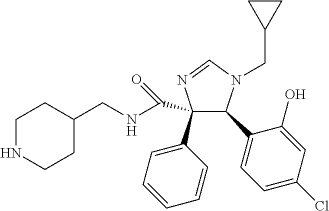

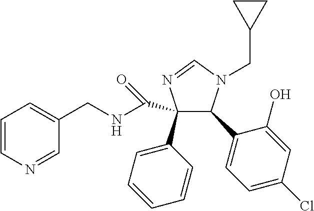

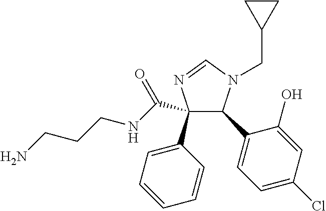

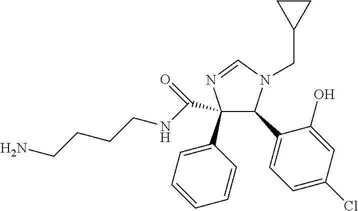

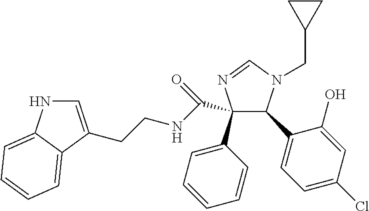

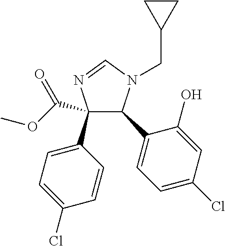

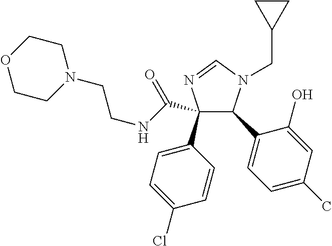

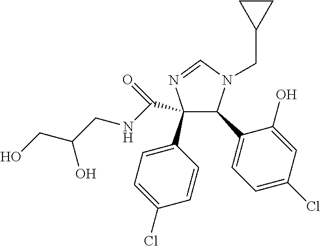

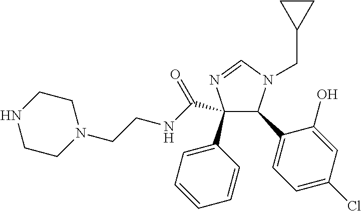

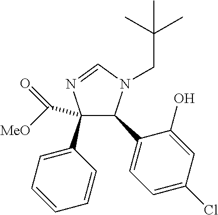

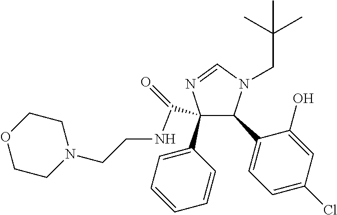

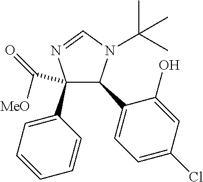

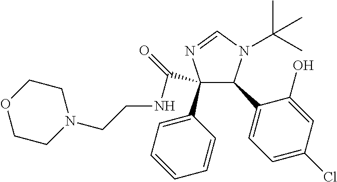

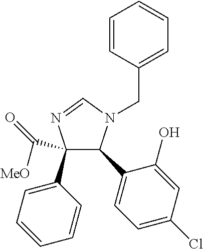

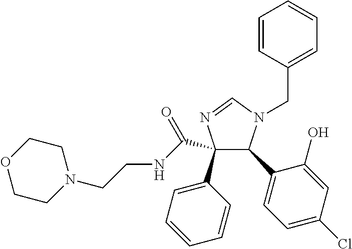

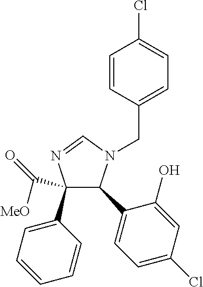

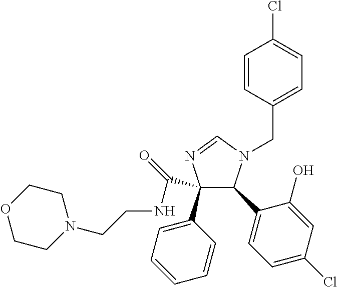

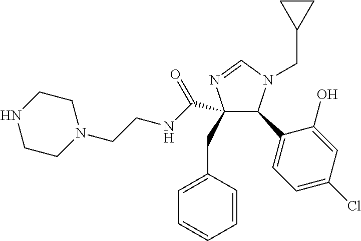

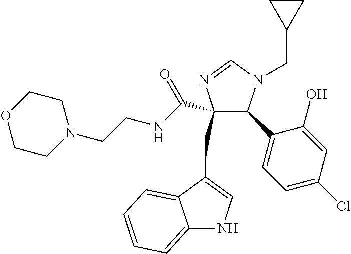

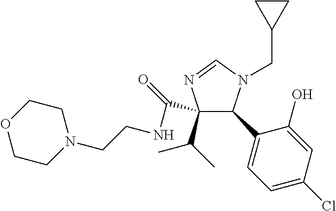

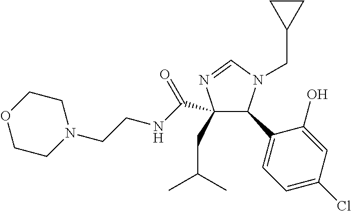

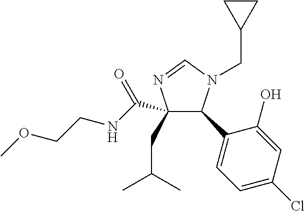

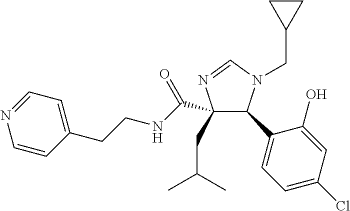

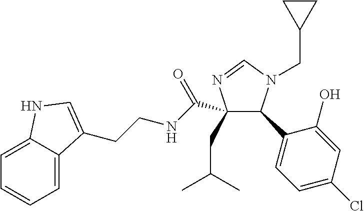

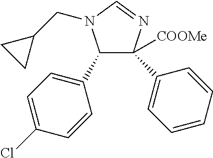

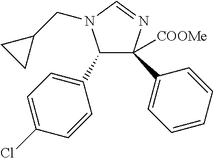

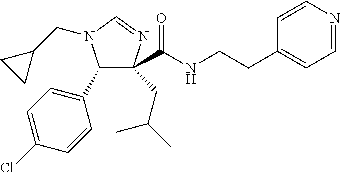

Compounds shown in Table 1 can exist as diasteromers, which were separated using variety of analytical techniques such as without limitation, column chromatography, high pressure liquid chromatography, crystallization or by the selective precipitation of a preferred diastereomer. If there is a discrepancy between a depicted structure and a name given to that structure, then the depicted structure controls. Additionally, if the stereochemistry of a structure or a portion of a structure is not indicated with, for example, by bold or dashed lines, the structure or portion of the structure is to be interpreted as encompassing all stereoisomers of it.

According to another embodiment, Formula I compounds, e.g., PB2 and PB11 (Table 1), were synthesized using the three-component Orru reaction involving an amine, an aldehyde, and a substituted α-cyanomethylacetate. D. van Leusen, et al., Org. React. 57: 417-666 (2001). The synthesis of compounds having other molecular scaffolds (see Table 1), likewise were accomplished using published protocols as illustrated below in Table 2.

TABLE 2

| no | reaction scheme | representative antagonist* |
|---|---|---|
| PB1 | 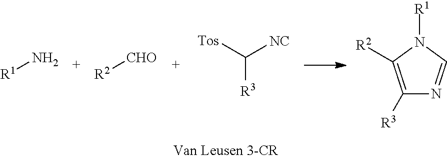 | 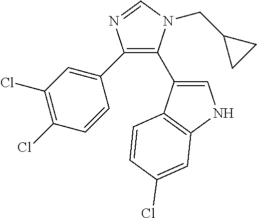 |
| PB2 | 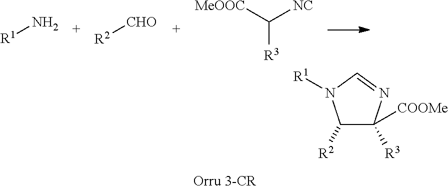 | 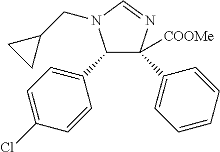 |
| PB3 | 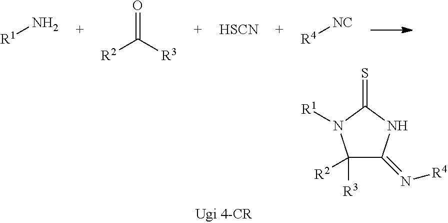 | 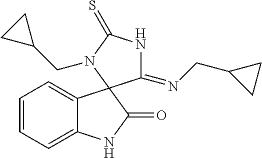 |
| PB4 | 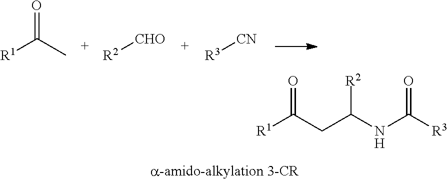 | 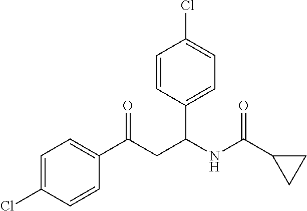 |
| PB5 | 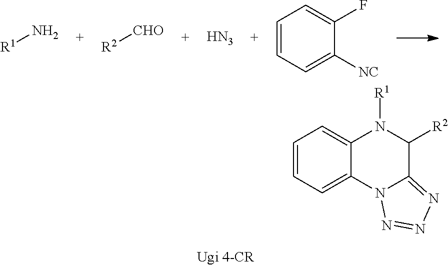 | 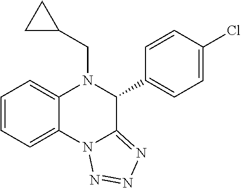 |
| PB6 | 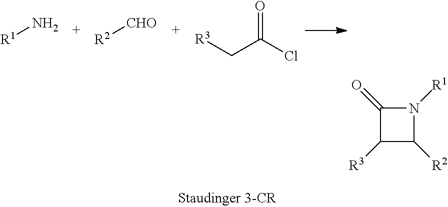 | 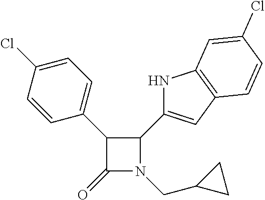 |
| PB7 | 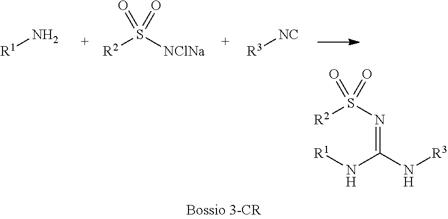 | 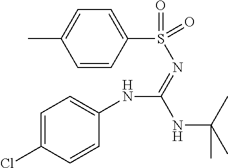 |
| PB8 | 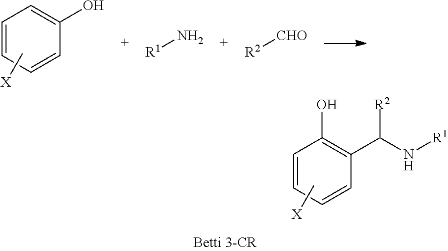 | 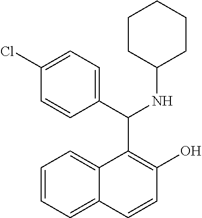 |
| PB9 | 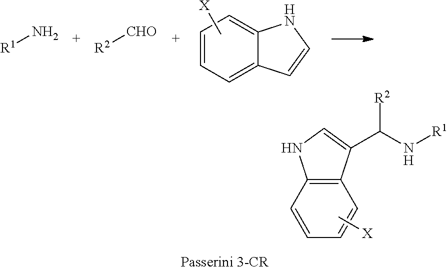 | 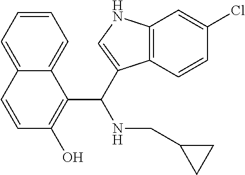 |
| PB10 | 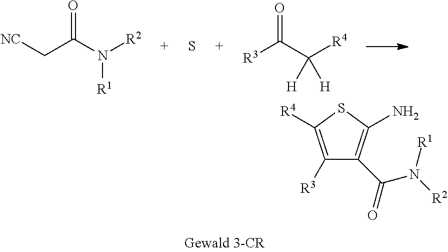 | 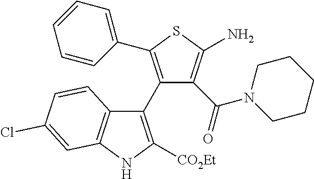 |

The affinities of imidazoline compounds of the present invention towards MDM2 or MDM4 proteins was measured by 2D H¹-N15 HSQC NMR spectroscopy. The present inventor chose NMR spectroscopy to test whether compounds in accordance with the present invention were antagonist of the p53/Hdm2 complex and were able to dissociate the preformed p53/Hdm2 complex. In addition, NMR spectroscopy was utilized to test the aqueous solubility of inventive compounds and to identify the position and identity of amino acid residues that are involved in binding. NMR spectral analysis was also used to study whether an inventive compound caused precipitation of p53 of hMDM2 proteins and to study whether pounds in accordance with the present invention upon binding caused protein conformational changes that were different from those caused by binding of p53 to hMDM2.

Typically, NMR samples contained 0.05-0.2 mM protein in 50 mM KH₂PO₄ and 50 mM Na₂HPO₄, pH 7.4, containing 150 mM NaCl and 5 mM DTT. Water suppression was carried out using the WATERGATE sequence. NMR spectra were acquired at 300 K on a Bruker DRX 600 MHz spectrometer equipped with a cryoprobe and the data was processed using the Bruker program Xwin-NMR V. 3.5.

NMR ligand binding experiments were carried out in an analogous way to those previously described. See Popowicz et al., Cell Cycle 6, 2386-92 (2007). For instance, 500 μL of the protein sample, at a concentration of about 0.1 mM, in 10% D₂O and a 20 mM stock solution of nutin-3 (purchased from Cayman Chemical, MI) in DMSO-d₆ were used in all experiments. The maximum concentration of DMSO at the end of titration was less than 1% and pH was maintained constant during the entire titration.

The inventive compounds were tested for binding to Hdm2 by performing a series of NMR titrations with isotopically enriched ¹⁵N-Hdm2. Strong binding of a compound to its target is indicated by appearance of splitting of the signals in a heteronuclear single quantum coherence (HSQC) spectrum, whereas a shift of signals indicates weaker binding. FIG. 1 shows ¹⁵N-HSQC spectra of Hdm2 titrated with PB14, a compound having an indole moiety as the anchor group and exemplifies spectral data showing a slow chemical exchange that is typical for compounds that bind hMDM2 tightly.

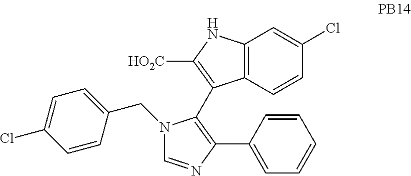

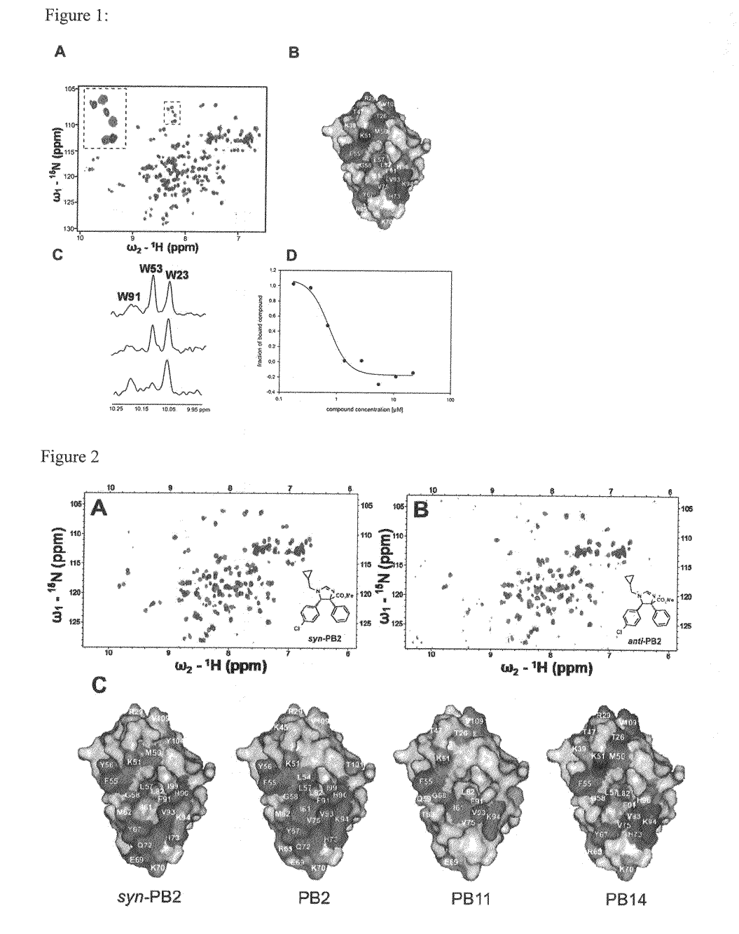

In contrast, as illustrated by FIG. 2, the ¹⁵N-HSQC of the anti-diastereomer of PB2 (below) shows a fast chemical exchange, indicating a weaker affinity of this compound for hMDM2.

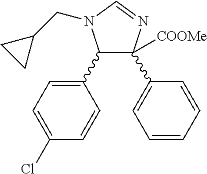

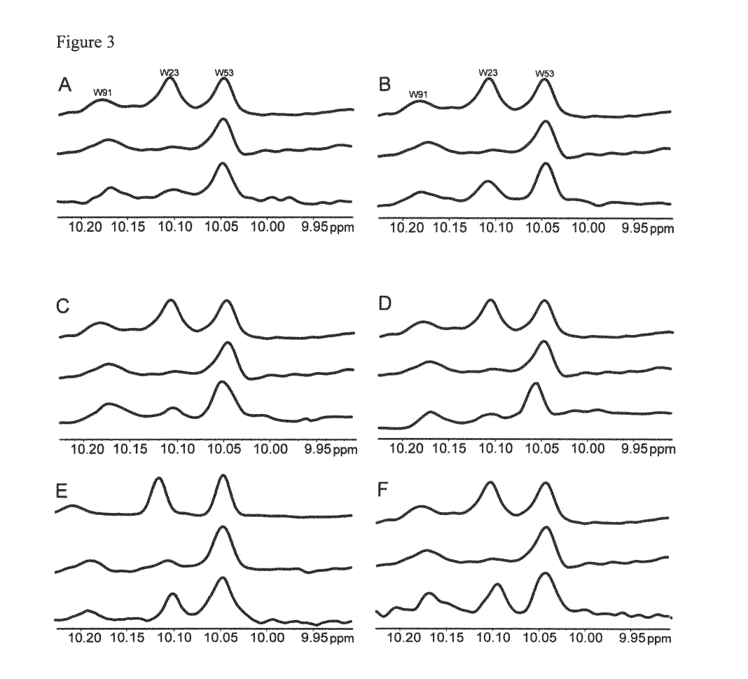

The ability of exemplary PB-compounds to disrupt p53-hMDM2 complex was determined using a ¹D-NMR antagonist-induced dissociation assay (AIDA), as illustrated by FIG. 3. See further discussion below.

Because of the demonstrated ability of the inventive compounds to disrupt or antagonize the p53/MDM2/MDM4 complex, the inventive imidazoline derivatives are candidate therapeutics for treating cancer as well as other cell proliferative diseases. In one embodiment, therefore, the invention provides a method for treating cancer in a subject comprising administering to the subject a compound as described herein. In the context of this invention, the terms “treat”, “treating” and “treatment” refer to the amelioration or eradication of a disease or symptoms associated with a disease. In certain embodiments, such terms refer to minimizing the spread or worsening of the disease resulting from the administration of compounds in accordance with this invention to a subject suffering from cancer.

Accordingly, the invention provides formulations of compounds belonging to Formula I, as candidate therapeutics for cancer. Inhibition of cancer is reflected by various biochemical indicia of tumor cell death, such as a reduction in tumor mass, a lowering of blood T-cell count, or the lowering of certain enzymes that are known to be up regulated in cancer, such as enzymes of the kinase family of proteins. Thus, the amount of compound that results in greater than about 50% decrease in one or more biochemical indicia of this disease in vitro can be used for determining an effective dose (“therapeutic dose”) in vivo. In one embodiment therefore a formulation that contains an amount of compound of Formula I that results in blood concentrations sufficient to cause at least a 50% decrease tumor cell death can be used as a starting point for treatment.

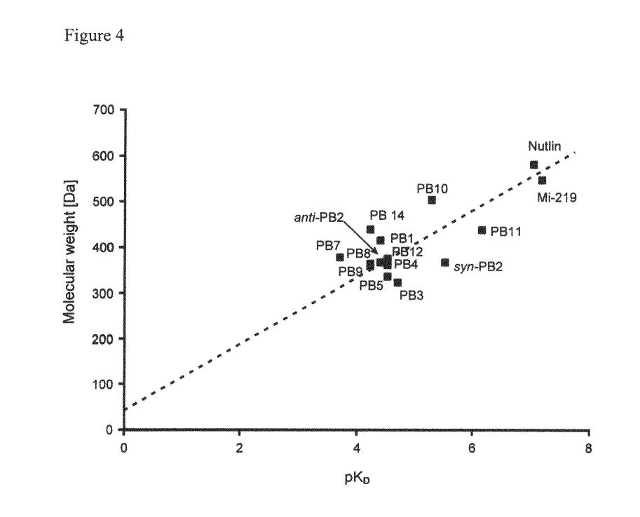

Because of the hydrophobic nature of the p53-hMDM2 binding region, many of the lead compounds showed poor aqueous solubility which is pharmaceutically undesirable because it presents a problem during formulation development and can reduce in vivo bioavailability of drug. The present inventor rationalized, therefore, that derivatization of an inventive compound to a more hydrophilic drugable molecule, for instance via amidation of a carbxylic acid group or the amidation of an ester group could potentially improve water solubility, binding affinity and bioavailability. See below for synthetic methodology. Indeed, for an exemplary imidazoline (PB2) according to the present invention, converting the carboxymethyl group to amide side chain gave the amide derivative PB11 that showed improved the potency and water solubility. See FIG. 4.

In one aspect, therefore, the present invention is directed to pharmaceutical formulations of Formula I compounds and the use of the inventive formulations to treat disease conditions associated with improper cell division activity, such as cancers. In one aspect, the present invention can provide combination therapy in which a patient or subject in need of therapy is administered a formulation of a Formula I compound in combination with one or more other compounds having similar or different biological activities.

In an aspect of the combination therapy routine, a therapeutically effective dose of inventive compound may be administered separately to a patient or subject in need thereof from a therapeutically effective dose of the combination drug. Moreover, the person of skill in the art will recognize that the two doses may be administered within hours or days of each other or the two doses may be administered together.

The invention also provides a pharmaceutical composition comprising one or more compounds according to Formula I or a pharmaceutically acceptable salt, solvate, stereoisomer, or prodrug, in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

According to one aspect of the invention, therefore, the pharmaceutical composition comprises a compound selected from those illustrated in Table 1 or a pharmaceutically acceptable salt, solvate, stereoisomer, or prodrug thereof, and a pharmaceutically acceptable carrier.

Improving the aqueous solubility permits formulation of the inventive compounds to be administered orally, or parenterally using unit dosage formulations. The term parenteral as used herein includes subcutaneous injections, intravenous, intramuscular, or infusion techniques.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

Inventive compositions suitable for oral use can be prepared according to any method known to the art for the manufacture of pharmaceutical compositions. For instance, liquid formulations of the inventive compounds contain one or more agents selected from the group consisting of sweetening agents, flavoring agents, coloring agents and preserving agents in order to provide pharmaceutically elegant and palatable preparations of the arginase inhibitor.

Formulations suitable for oral administration are known and include without limitation tablets, troches, lozenges, aqueous or oily suspensions, dispersible powders or granules, emulsion, hard or soft capsules, syrups or elixirs.

For Formula I compounds, esterification of the C-4 carboxylate group to form the resultant prodrug provides an acceptable alternative route for delivering these charged compounds into cells. Because the compounds target intracellular MDM proteins in cancer cells, any route of administration resulting in sustained blood concentrations sufficient to penetrate such cells should produce a therapeutic benefit.

### Synthesis of Compounds

Compounds of the invention are prepared using any number of the published methodologies as further described herein below. The choice of an appropriate synthetic methodology is guided by the choice of compound desired and the nature of functional groups present in the intermediate and final product. Thus, selective protection/deprotection protocols may be necessary during synthesis depending on the specific functional groups desired and protecting groups being used. A description of such protecting groups and how to introduce and remove them is found in PROTECTIVE GROUPS IN ORGANIC SYNTHESIS (3^{rd} ed.), John Wiley and Sons, New York (1999).

1H- and 13C-NMR spectra were recorded at 300 K on a Bruker Avance II Ultrashield Plus 600 at 600 and 150 MHz, respectively. Chemical shift values are in ppm relative to residual solvent signal.

Typically, NMR samples contained 0.05-0.2 mM protein in 50 mM KH₂PO₄ and 50 mM Na₂HPO₄, pH 7.4, containing 150 mM NaCl and 5 mM DTT. Water suppression was carried out using the WATERGATE sequence. NMR data were processed using the Bruker program Xwin-NMR version 3.5. NMR ligand binding experiments were carried out in an analogous way to those previously described. See D'Silva, et. al., JACS., 127(38): 13220-13226, (2005) and Popowicz, et. al., Cell Cycle, 6(19): 2386-2392, (2007).

For example, 500 μL of the protein sample, at a concentration of about 0.1 mM, in 10% D₂O and a 20 mM stock solution of nutin-3 (purchased from Cayman Chemical, MI) in DMSO-d₆ were used in all of the experiments. The maximum concentration of DMSO at the end of titration experiments was less than 1%. The pH was maintained constant during the entire titration. The ¹H-¹⁵N-HSQC spectra were recorded using fast HSQC pulse sequence as described by Mori et al., J. Magn. Reson., 108: 94-98, (1995). The maximum concentration of DMSO at the end of titration experiments was less than 1%.

Flash chromatography was performed with the indicated solvent mixture on silica gel, MP Silitech 32-63 D, 60 Å, Bodman. Chromatotron chromatography was performed on Harrison Research Chromatotron, Ser. no. 65F with the indicated solvent mixture using silica gel, Merck, TLC grade 7749, with gypsum binder and fluorescent indicator, Sigma-Aldrich. Thin layer chromatography was performed using Whatmann flexible-backed TLC plates on aluminum with fluorescence indicator. Compounds on TLC were visualized by UV-detection. HPLC-MS measurements were done on a Shimadzu prominence HPLC equipped with a dual wavelength UV detector and an API 2000 LC-MS/MS system, Applied Biosystems MDS SCIEX, (MS) using a Dionex Acclaim 120 column (C18, 3 μm, 120 Å, 2.1×150 mm) using a mobile phase of water and acetonitrile, both containing 0.1% acetic acid and the following gradient: 5-90% acetonitrile in 7 min, injection volume: 5 μL, detection wavelength 254 nm. HRMS measurements were performed at the Department of Chemistry, University of Pittsburgh with a Waters/Micromass Q-T of spectrometer, ionization mode: ESI. Microwave reactions were performed on the Emrys Optimizer system from Personal Chemistry.

### EXAMPLES

### PB1: 6-Chloro-3-[3-cyclopropylmethyl-5-(3,4-dichlorophenyl)-3H-imidazol-4-yl]-1H-indole

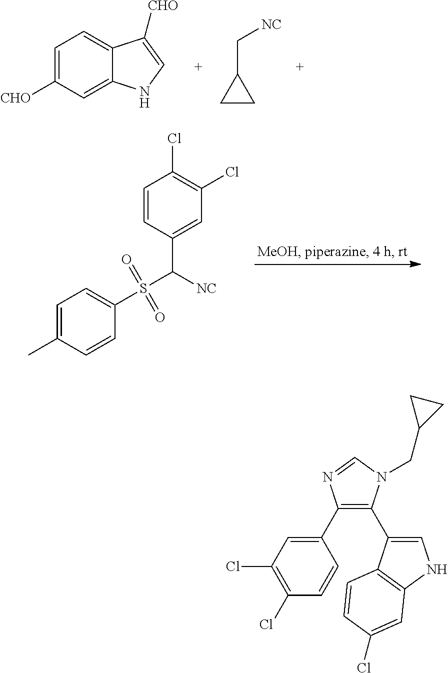

6-Chloro-1H-indole3-carbaldehyde (180 mg, 1 mmol) were dissolved in 2 mL of MeOH, and 85.6 μL (1 mmol) cyclopropylmethyl amine was added dropwise. The reaction mixture was stirred for 4 h at room temperature and 1,2-dichloro-4-[isocyano(toluene-4-sulfonyl)methyl]benzene (340 mg, 1 mmol) and piperazine (86 mg, 1 mmol) were added and stirred over night at room temperature. The solvent was evaporated and the crude product purified by chromatography on silica with a gradient of 3:1 to 2:1 heptane/ethyl acetate to yield 6-chloro-3-[3-cyclopropylmethyl-5-(3,4-dichlorophenyl)-3H-imidazol-4-yl]-1H-indole (PB1) 356 mg (86%); ¹H-NMR (CDCl₃, 600 MHz): δ 0.17 (d, J=4.80 Hz, 2H), 0.54 (d, J=7.86 Hz, 2H), 0.99-1.03 (m, 1H), 3.59 (d, J=6.90 Hz, 2H), 7.07-7.12 (m, 2H), 7.17-7.21 (m, 2H), 7.25-7.26 (m, 1H), 7.46 (m, 1H), 7.74-7.75 (m, 1H), 7.86 (s, 1H), 9.63 (s, 1H); ¹³C-NMR (CDCl₃, 150 MHz): δ 4.0, 11.2, 49.8, 104.5, 111.4, 120.0, 131.2, 131.6, 124.8, 125.4, 125.6, 127.5, 128.5, 129.5, 129.7, 131.8, 134.5, 136.3, 136.6, 137.1; HPLC-MS (ESI): rₜ=9.53 min, m/z 416 [M+H]⁺; HRMS (ESI-TOF) C₂₁H₁₇Cl₃N₃ m/z calcd [M+H]⁺416.0488, found 416.0498.

### PB2: Methyl 5-(4-chlorophenyl)-1-cyclopropylmethyl-4-phenyl-4,5-dihydro-1H-imidazole-4-carboxylate

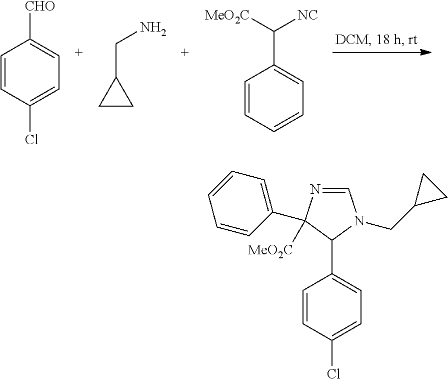

p-Chlorobenzaldehyde (422 mg, 3 mmol) was solubilized in 20 ml dry dichloromethane. Cyclopropylmethylamine (257 μL, 3 mmol) and methyl isocyanophenylacetate (525 mg, 3 mmol) were added and the mixture was allowed to stir over night at room temperature. Isolation of the mixture of the two diasteromers by column chromatography on silica gel with a gradient of 3:1 to 1:5 petroleum ether/ethyl acetate gradient yielded 893 mg (81%) of methyl 5-(4-chlorophenyl)-1-cyclopropylmethyl-4-phenyl-4,5-dihydro-1H-imidazole-4-carboxylate (PB2) as a yellow oil as a mixture of diastereomers (33:17). The mixture of the two diastereomers (760 mg) was separated by column chromatography on neutral alumia with ethyl acetate to give 260 mg of pure major diastereomer and 374 mg of the mixture of two diastereomers. ¹H-NMR for the major diastereomer (CDCl₃, 600 MHz): δ 0.05-0.06 (m, 2H), 0.47-0.51 (m, 1H), 0.59-0.62 (m, 1H), 0.88-0.91 (m, 1H), 2.55-2.59 (m, 1H), 3.09-3.12 (m, 1H), 3.79 (s, 3H), 5.64 (s, 1H), 6.90-6.91 (m, 4H), 7.04-7.06 (m, 5H), 7.44 (s, 1H); ¹³C-NMR for the major diastereomer (CDCl₃, 150 MHz): δ 2.4, 4.5, 9.1, 50.0, 52.7, 68.7, 84.1, 126.2, 126.8, 127.3, 127.5, 132.7, 134.1, 136.9, 156.3, 173.8; ¹H-NMR for the minor diastereomer (CDCl₃, 600 MHz): δ 0.02-0.04 (m, 2H), 0.44-0.46 (m, 1H), 0.54-0.56 (m, 1H), 0.82-0.84 (m, 1H), 2.61-2.65 (m, 1H), 3.09-3.13 (m, 1H), 3.29 (s, 3H), 5.09 (s, 1H), 7.31-7.43 (m, 8H), 7.76-7.77 (m, 2H); ¹³C-NMR for the minor diastereomer (CDCl₃, 150 MHz): δ 2.5, 4.3, 9.3, 49.7, 51.7, 72.8, 85.0, 126.3, 127.4, 128.0, 128.3, 129.0, 133.8, 135.3, 143.2, 155.1, 170.7; HPLC-MS (ESI): rₜ=12.13 min m/z 369 [M+H]⁺; HRMS (ESI-TOF) m/z calcd for C₂₁H₂₂ClN₂O₂ [M+H]⁺369.1370, found 369.1365.

### PB3: (Z)-3-(cyclopropylmethyl)-5-(cyclopropylmethylimino)-2-thioxospiro (imidazolidine-4,3′-indolin)-2′-one

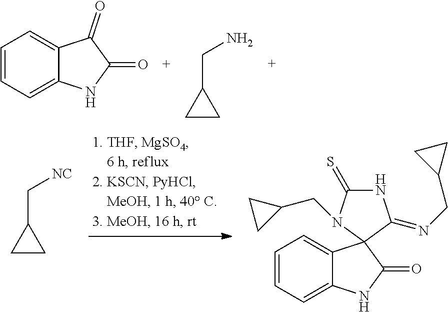

Isatin (908 mg, 5 mmol) and cyclopropylmethylamine (431 μL, 5 mmol) were dissolved in THF. A small amount MgSO₄ was added and the reaction mixture was refluxed for 6 h, filtered and evaporated to give the precondensed Schiff base. A solution of KSCN (485 mg, 5 mmol) and pyridium hydrochloride (528 mg, 5 mmol) in 15 mL of MeOH was heated at 40° C. for 1 h, then cooled with ice-water and filtered. The Schiff base (1.0 g, 5 mmol) was added to the solution and isocyanomethylcyclopropane (405 mg, 5 mmol) was added drop wise. The reaction mixture was stirred at room temperature overnight. The solvent was evaporated and the residue purified by column chromatography to yield (Z)-3-(cyclopropylmethyl)-5-(cyclopropylmethylimino)-2-thioxospiro(imidazolidine-4,3′-indolin)-2′-one (PB3) 350 mg (25%); ¹H-NMR for the major diastereomer (MeOD, 600 MHz): δ −0.10 (m, 1H), −0.01 (m, 1H), 0.22 (m, 2H), 0.27 (m, 1H), 0.35 (m, 1H), 0.46 (m, 2H), 0.81 (m, 1H), 1.03 (m, 1H), 3.18 (dd, J=7.2, 14.4 Hz, 1H), 3.23 (dd, J=1.2, 6.6 Hz, 2H), 3.70 (dd, J=6.6, 14.4 Hz, 1H), 7.07 (d, J=7.8 Hz, 1H), 7.14 (m, 2H), 7.45 (m, 1H); ¹³C-NMR (MeOD, 150 MHz): δ 2.3, 2.4, 3.2, 4.2, 9.5, 9.6, 49.4, 76.9, 111.3, 123.2, 123.3, 124.9, 131.4, 143.3, 171.3, 173.4, 197.7; HPLC-MS (ESI): rₜ=8.25 min m/z 341 [M+H]⁺; HRMS (ESI-TOF) m/z calcd for C₁₈H₂₀N₄OS 340.1357, found 340.1366.

### PB4: Cyclopropanecarboxylic acid [1,3-bis-(4-chlorophenyl)-3-oxopropyl]amide

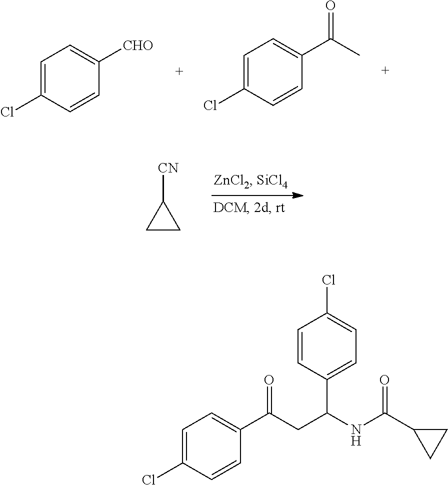

141 mg (1 mmol) 4-Chlorobenzaldehyde, 132.6 μL (1 mmol) 1-(4-chlorophenyl)ethanone and 75.6 μL (1 mmol) cyclopropanecarbonitrile were combined in dry DCM. Zinc chloride (273 mg, 2 eq, 2 mmol) and silicon tetrachloride (458.3 μL, 4 eq, 4 mmol) were added and the reaction mixture was allowed to stir for 2 days at room temperature. The reaction mixture was purified by chromatography on silica gel with 4:1 heptane/ethyl acetate to yield cyclopropanecarboxylic acid [1,3-bis-(4-chlorophenyl)-3-oxopropyl]amide (PB4) 43 mg (12%); ¹H-NMR (CDCl₃, 600 MHz): δ 0.77-0.79 (m, 2H), 0.98-1.00 (m, 2H), 1.40-1.45 (m, 1H), 3.39-3.43 (m, 1H), 3.73-3.77 (m, 1H), 5.53-5.56 (m, 1H), 6.80 (d, 1H), 7.28-7.33 (m, 4H), 7.44-7.47 (m, 2H), 7.86 (d, 2H); ¹³C-NMR (CDCl₃, 150 MHz): δ 7.4, 7.5, 14.9, 43.1, 49.5, 127.9, 128.8, 129.1, 129.5, 133.3, 134.8, 139.4, 140.2, 173.1, 197.2; HPLC-MS (ESI): rₜ=11.30 min m/z 362 [M+H]⁺; HRMS (ESI-TOF) m/z calcd for C₁₉H₁₇Cl₂NO₂Na [M+Na]⁺384.0534, found 384.0550.

### PB5: 4-(4-Chlorophenyl)-5-cyclopropylmethyl-4,5-dihydro-1,2,3,5,9b-pentaazacyclopenta[α]naphthalene

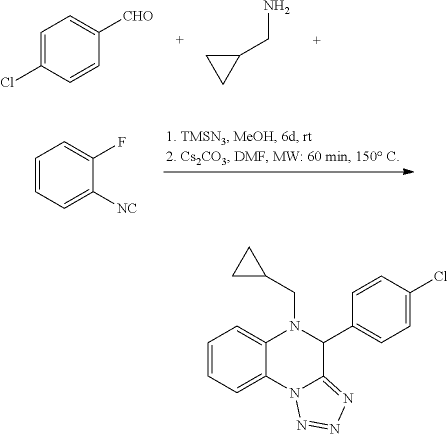

4-Chlorobenzaldehyde (422 mg, 3 mmol) and cyclopropylmethylamine (262.64, 3 mmol) were dissolved in 3 mL of MeOH and stirred for 5 h at room temperature. 1-Fluoro-2-isocyanobenzene (472 mg, 1.3 eq, 3.9 mmol) was added and the reaction mixture was allowed to stir for 6 days at room temperature. The solvent was evaporated and the residue dissolved in ethyl acetate and washed with water and brine. The organic layer was dried over MgSO₄ and concentrated. The crude product was purified by chromatography on silica gel with a 9:1 to 4:1 gradient of heptane/ethyl acetate to yield 1074 mg of {(4-chlorophenyl)-[1-(2-fluorophenyl)-1H-tetrazol-5-yl]methyl}cyclopropylmethylamine. {(4-Chlorophenyl)-[1-(2-fluorophenyl)-1H-tetrazol-5-yl]methyl}cyclopropylmethylamine (100 mg, 0.28 mmol) was dissolved in 4 mL of dry DMF and baked Cs₂CO₃ (182 mg, 2 eq, 0.56 mmol) was added and the reaction mixture was heated in the microwave for 60 min at 150° C. The solvent was evaporated and the residue dissolved in ethyl acetate and extracted with water and brine. The organic layer was dried over MgSO₄, filtered and evaporated. The crude product was purified by chromatography on silica gel with 4:1 heptane/ethyl acetate to yield 4-(4-chlorophenyl)-5-cyclopropylmethyl-4,5-dihydro-1,2,3,5,9b-pentaazacyclopenta[α]naphthalene (PB5) 19 mg (20%); ¹H-NMR (CDCl₃, 600 MHz): δ 0.15-0.23 (m, 2H), 0.53-0.57 (m, 1H), 0.65-0.68 (m, 1H), 1.04-1.06 (m, 1H), 3.01-3.05 (m, 1H), 3.57-3.60 (m, 1H), 6.50 (s, 1), 7.00-7.05 (m, 2H), 7.22-7.31 (m, 4H), 7.37-7.40 (m, 1H), 7.99-7.00 (d, 1H); HPLC-MS (ESI): rₜ=12.29 min m/z 337 [M+H]⁺; HRMS (ESI-TOF) m/z calcd for C₁₈H₁₆ClN₅ 337.1094, found 337.1093.

### PB6: 4-(6-Chloro-1H-indol-2-yl)-3-(4-chlorophenyl)-1-cyclopropylmethylazetidin-2-one

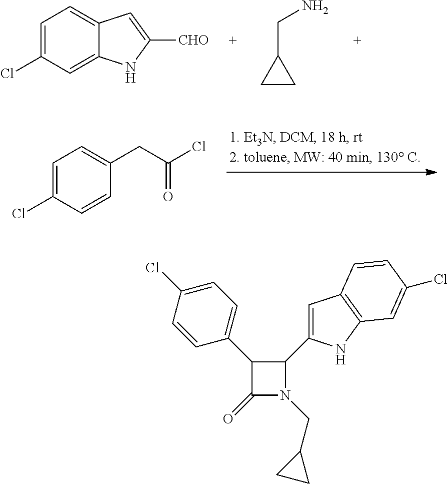

6-Chloro-1H-indole-2-carbaldehyde (180 mg, 1 mmol) and cyclopropylmethylamine (85.6 mL, 1 mmol) were dissolved in DCM. A small amount MgSO₄ was added and the mixture was stirred over night. The salt was filtered off and the filtrate concentrated under reduced pressure. The residue was dissolved in toluene, and triethylamine (669 μL, 4.8 mmol) and (4-chlorophenyl)acetylchloride (251.6 μL, 1.72 mmol) were added simultaneously. The reaction mixture was heated in the microwave for 40 min at 130° C. After the mixture cooled to room temperature the solid was filtered off and the filtrate was evaporated. The residue was dissolved in ethyl acetate and extracted with water and brine. The organic layer was dried over MgSO₄, filtered and evaporated. The crude product was purified by chromatography on silica gel with 4:1 heptane/ethyl acetat to yield 4-(6-chloro-1H-indol-2-yl)-3-(4-chlorophenyl)-1-cyclopropylmethylazetidin-2-one (PB6) 66 mg (18%); ¹H-NMR (CDCl₃, 600 MHz): δ 0.43-0.45 (m, 2H), 0.51-0.52 (m, 2H) 0.92-0.95 (m, 1H), 2.65-2.69 (m, 1H), 3.52-3.56 (m, 1H), 4.43 (s, 1H), 4.79 (s, 1H), 6.52 (s, 1H), 7.07-7.08 (m, 1H), 7.24-7.48 (m, 6H), 9.98 (s, 1H); ¹³C-NMR (CDCl₃, 150 MHz): δ 3.0, 4.2, 9.3, 45.9, 58.2, 62.2, 103.5, 111.3, 120.8, 121.3, 126.3, 128.5, 128.6, 129.3, 133.0, 133.9, 134.6, 137.5, 168.1; HPLC-MS (ESI): rₜ=12.54 min m/z 384 [M−H]⁻; HRMS (ESI-TOF) m/z calcd for C₂₁H₁₈Cl₂N₂O 384.0796, found 384.07977.

### PB7: N-[1-tert-Butylamino-1-(4-chlorophenylamino)meth-(Z)-ylidene]-4-methylbenzene-sulfonamide [11]

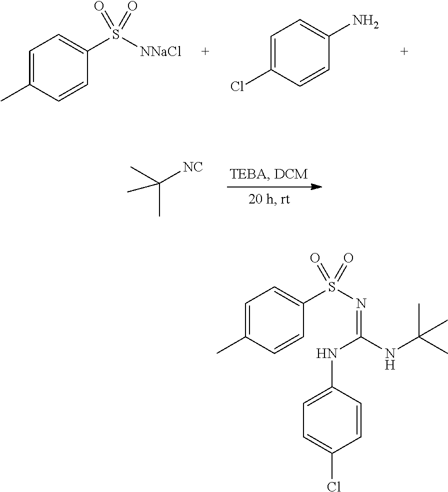

A solution of chloramine T (228 mg, 1 mmol), 4-chlorophenylamine (128 mg, 1 mmol) and tert-butylisocyanide (83 mg, 1 mmol) in 5 mL of dry DCM was treated with benzyltriethylammonium chloride (5 mg) and stirred for 20 h at room temperature. The reaction was quenched with water and the organic layer was separated, dried over Na₂SO₄, filtered and evaporated. The crude product was purified by chromatography on silica with 2:1 petroleum ether/ethyl acetate to yield N-[1-tert-butylamino-1-(4-chlorophenylamino)meth-(Z)-ylidene]-4-methylbenzenesulfonamide (PB7) 94 mg (24%); ¹H-NMR (CDCl₃, 600 MHz): δ 1.32 (s, 9H), 2.43 (s, 3H), 6.62 (d, 1H), 7.07-7.10 (m, 2H), 7.28 (d, 2H), 7.38 (d, 2H), 7.84 (d, 2H), 8.81 (bs, 1H); ¹³C-NMR (CDCl₃, 150 MHz): δ 21.5, 29.2, 52.8, 116.3, 125.9, 126.4, 127.0, 129.1, 129.3, 130.3, 134.4, 140.7, 142.1, 145.1, 152.7; HPLC-MS (ESI-TOP): rₜ=11.75 min m/z 380 [M+H]⁺; HRMS (ESI-TOF) m/z calcd for C₁₈H₂₃ClN₃O₂S [M+H]⁺380.1200, found 380.1189.

### PB8: 1-[(4-chlorophenyl)(cyclohexylamino)methyl]naphthalen-2-ol

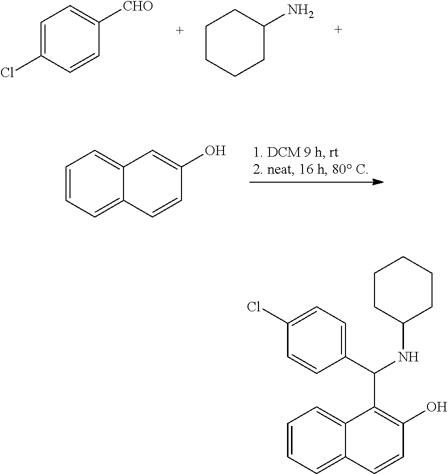

4-Chlorobenzaldehyde (337 mg, 1.2 eq, 2.4 mmol) and cyclohexylamine (239.9 μL, 1.05 eq, 2.1 mmol) were diluted in DCM and stirred for 9 h at room temperature. The solvent was evaporated and the precondensed Schiff base was combined with naphthalen-2-ol (288 mg, 1 eq, 2 mmol) and heated to 80° C. for 15 h. The reaction mixture was purified by chromatography on silica gel with 4:1 heptane/ethyl acetate to yield 1-[(4-chlorophenyl)cyclohexylaminomethyl]naphthalen-2-ol (PB8) 343 mg (47%); ¹H-NMR (CDCl₃, 600 MHz): δ 0.71-0.82 (m, 2H), 1.06-1.22 (m, 3H), 1.50-1.51 (m, 1H), 1.51-1.53 (m, 1H), 1.58-1.61 (m, 1H), 1.86-1.88 (m, 1H), 2.15-2.17 (m, 1H), 2.61 (bs, 1H), 5.76 (s, 1H), 7.06-7.07 (d, 1H), 7.15-7.19 (m, 3H), 7.25-7.28 (m, 3H), 7.56-7.58 (d, 1H), 7.64-7.66 (m, 2H), 13.88 (bs, 1H); ¹³C-NMR (CDCl₃, 150 MHz): δ 24.6, 24.7, 25.5, 32.4, 33.2, 55.5, 59.6, 113.2, 120.1, 120.6, 122.2, 126.3, 128.3, 128.7, 129.5, 131.9, 133.5, 140.4, 157.2; HPLC-MS (ESI-TOF): rₜ=10.43 min m/z 366 [M+H]⁺; HRMS (ESI-TOF) m/z calcd for C₂₃H₂₄ClNO 365.1546, found 365.1549.

### PB9: [(6-Chloro-1H-indol-3-yl)naphthalen-1-yl-methyl]cyclopropylmethylamine

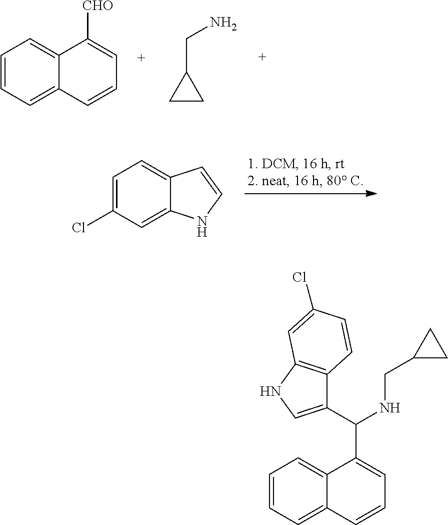

Naphthalene-1-carbaldehyde (327 μL, 1.2 eq, 2.4 mmol) and cyclopropylmethylamine (180 μL, 1.05 eq, 2.1 mmol) were diluted in DCM and stirred over night at room temperature. The solvent was evaporated and the precondensed Schiff base was combined with 6-chloro-1H-indole (303 mg, 1 eq, 2 mmol) and heated to 80° C. for 15 h. The reaction mixture was purified by chromatography on silica gel with 4:1 heptane/ethyl acetate to yield [(6-chloro-1H-indol-3-yl)naphthalen-1-ylmethyl]cyclopropylmethylamine (PB9) 270 mg (37%); ¹H-NMR (CDCl₃, 600 MHz): δ 0.08 (m, 2H), 0.45 (m, 2H), 1.03-1.05 (m, 1H), 2.44 (bs, 1H), 2.57-2.64 (m, 2H), 5.95 (s, 1H), 6.66 (s, 1H), 7.03 (d, 1H), 7.16 (s, 1H), 7.37-7.45 (m, 3H), 7.55 (d, 1H), 7.74-7.75 (m, 2H), 7.76 (d, 1H), 7.85 (d, 1H), 8.06 (s, 1H); ¹³C-NMR (CDCl₃, 150 MHz): δ 3.5, 3.7, 11.4, 53.7, 55.1, 111.3, 118.9, 119.9, 120.3, 123.3, 123.9, 124.4, 125.1, 125.5, 125.6, 126.0, 127.9, 128.9, 131.5, 134.1, 136.8, 138.3; HPLC-MS (ESI-TOF): rₜ=9.34 min m/z 359 [M]⁻; HRMS (ESI-TOF) m/z calcd for C₂₃H₂₁ClN₂ 360.1393, found 360.1401.

### PB10: Ethyl 3-(5-amino-2-phenyl-4-(piperidine-1-carbonyl)thiophen-3-yl)-6-chloro-1H-indole-2-carboxylate

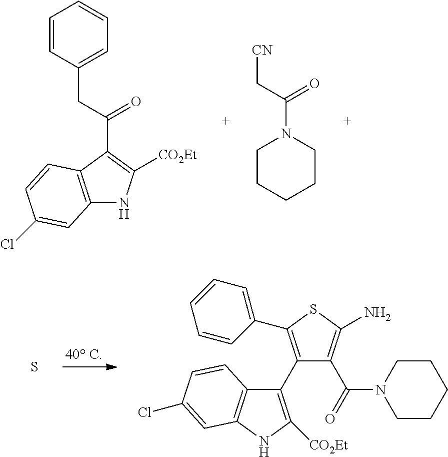

Titanium tetrachloride (2.0 mL, 1.0 M in toluene) was added dropwise to a solution of ethyl 6-chloro-3-(2-phenylacetyl)-1H-indole-2-carboxylate (1.0 mmol, 340 mg), 3-oxo-3-(piperidin-1-yl)propanenitrile (1.5 mmol, 228 mg) in 1 mL of THF. Then triethylamine (0.3 mL) was added dropwise, the mixture was stirring under 40° C. overnight. After work up with 10% HCl, the mixture was extracted by ethyl acetate. The combined organic layer was washed with 2 M NaOH, the dried over magnesium sulfate. The intermediate was purified by column chromatography on silica gel (petroleum ether/ethyl acetate, 10:1 to 5:1) as yellow oil (160 mg, yield: 34%, a mixture of Z- and E-isomers). Then the isolated intermediate was treated with sulfur (32 mg), triethylamine (0.15 mL) in 1 mL of ethanol and the mixture was stirring under 50° C. for 2 days. The product was purified by column chromatography on silica gel (petroleum ether/ethyl acetate, 5:1) as brown solids (14 mg, yield: 8%). ¹H-NMR (CDCl₃, 600 MHz): δ 9.14 (1H, s), 7.38 (1H, s), 6.98-7.08 (7H, m), 4.73 (2H, br.s), 4.01-4.17 (2H, m), 3.06-3.18 (4H, m), 1.23-1.27 (6H, m), 1.17 (3H, t, J=7.2 Hz); ¹³C-NMR (CDCl₃, 150 MHz): δ 14.0, 24.2, 29.3, 56.0, 61.1, 111.4, 116.8, 117.1, 122.1, 123.0, 126.5, 127.8, 128.2, 134.5, 135.7, 152.9, 160.7, 161.4; HPLC-MS (ESI-TOF): rₜ=11.46 min m/z 508.0 [M+H]⁺; HRMS (ESI-TOF) m/z calcd for C₂₇H₂₇N₃O₃SCl [M+H]⁺, calcd 508.1462, found 508.1425.

### PB11: cis-5-(4-chlorophenyl)-1-(cyclopropylmethyl)-4-isobutyl-N-(2-(pyridin-4-yl)ethyl)-4,5-dihydro-1H-imidazole-4-carboxamide

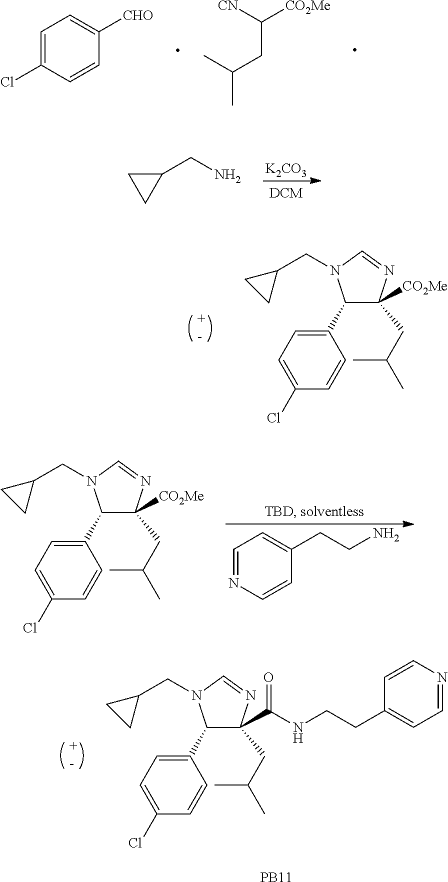

Synthetic procedure as recently described by Srivastava et al., J. Comb. Chem., 11: 631-639 (2009). Yield 35 mg (0.1 mmol, 79%); ¹H-NMR (CDCl₃, 600 MHz): δ −0.02-0.03 (2H, m), 0.43-0.48 (1H, m), 0.53-0.58 (1H, m), 0.66 (3H, d, J=6 Hz), 0.76-0.83 (4H, m), 0.89 (1H, dd, J=12 Hz & 6 Hz), 1.10 (1H, dd, J=12 Hz & 6 Hz), 1.53-1.60 (1H, m), 2.62 (1H, dd, J=12 Hz & 6 Hz), 2.84-2.96 (2H, m), 3.09 (1H, dd, J=12 Hz & 6 Hz), 3.56-3.68 (2H, m), 4.84 (1H, s), 7.16-7.18 (3H, m), 7.24-7.30 (2H, m), 7.34 (2H, brd, J=12 Hz), 8.53 (2H, brd, J=6 Hz); ¹³C-NMR (CDCl₃, 150 MHz): 5.69, 4.77, 9.33, 23.73, 24.40, 24.61, 29.70, 35.05, 39.26, 45.05, 50.32, 70.07, 79.84, 124.13, 128.45, 133.53, 134.20, 147.94, 149.86, 155.54, 176.39; HRMS (ESI-TOF) C₂₅H₃ClN₄O m/z calcd [M+H]⁺439.2265, found 439.2237 [M+H]⁺.

### PB12: N-[1,3-Bis-(4-chlorophenyl)-3-oxopropyl]-2-cyclopropylacetamide

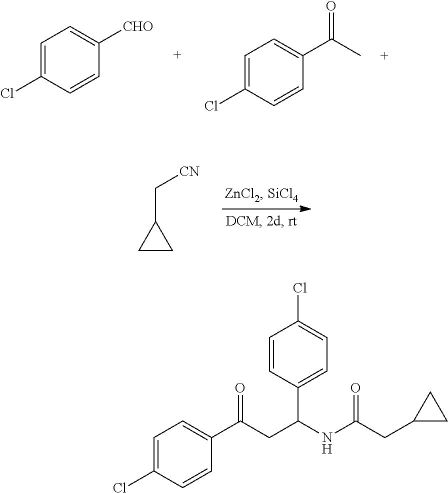

4-Chlorobenzaldehyde (141 mg, 1 mmol), 1-(4-chlorophenyl)ethanone (132.6 μL, 1 mmol) and cyclopropyl acetonitrile (93.9 μL, 1 mmol) were combined in dry DCM. Zinc chloride (273 mg, 2 eq, 2 mmol) and silicon tetrachloride (458.3 μL, 4 eq, 4 mmol) were added and the reaction mixture was stirred for 2 days at room temperature. The reaction mixture was purified by chromatography on silica gel with 4:1 heptane/ethyl acetate to yield N-[1,3-bis-(4-chlorophenyl)-3-oxopropyl]-2-cyclopropylacetamide (PB11) 71 mg (19%); ¹H-NMR (CDCl₃, 600 MHz): δ 0.22-0.25 (m, 2H), 0.66-0.69 (m, 2H), 0.97-1.05 (m, 1H), 2.21 (d, 2H), 3.39-3.43 (m, 1H), 3.73-3.77 (m, 1H), 5.55-5.59 (m, 1H), 7.22 (d, 1H), 7.28-7.31 (m, 4H), 7.44-7.46 (m, 2H), 7.85-7.87 (m, 2H); ¹³C-NMR (CDCl₃, 150 MHz): δ 4.4, 4.6, 7.1, 41.6, 42.9, 49.1, 125.5, 127.1, 127.9, 128.5, 128.7, 128.8, 129.1, 129.5, 133.3, 134.8, 139.5, 140.2, 172.0, 197.3; HPLC-MS (ESI-TOF): rₜ=11.49 min m/z 376 [M+H]⁺; HRMS (ESI-TOF) m/z calcd for C₂₀H₁₉Cl₂NO₂Na [M+Na]⁺ 398.0691, found 398.0678.

### PB13: 7-Bromo-1-[(4-chlorophenyl)(cyclopropylmethylamino) methyl]naphthalen-2-ol

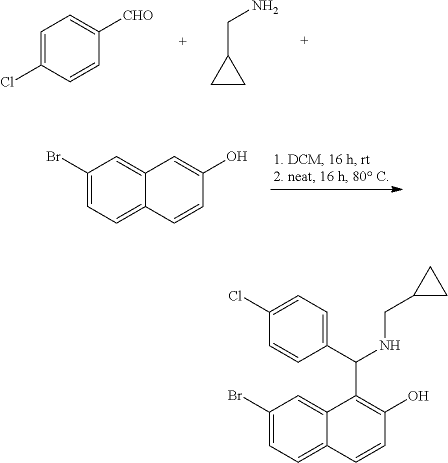

4-Chlorobenzaldehyde (337 mg, 1.2 eq, 2.4 mmol) and cyclopropylmethyl amine (180.1 μL, 1.05 eq, 2.1 mmol) were diluted in DCM and stirred over night at room temperature. The solvent was evaporated and the precondensed Schiff base was combined with 7-bromo-naphthalen-2-ol (444 mg, 1 eq, 2 mmol) and heated to 80° C. for 15 h. The reaction mixture was purified by chromatography on silica gel with heptane/ethyl acetate 4:1 to yield 7-bromo-1-[(4-chlorophenyl)(cyclopropylmethylamino)methyl]naphthalen-2-ol (PB12) 320 mg (38%); ¹H-NMR (CDCl₃, 600 MHz): δ 0.13-0.19 (m, 1H), 0.19-0.21 (m, 1H), 0.51-0.56 (m, 2H), 1.02-1.05 (m, 1H), 2.55-2.58 (m, 1H), 2.71-2.74 (m, 1H), 5.58 (s, 1H), 7.13 (d, 1H), 7.25-7.7.31 (m, 3H), 7.37-7.39 (m, 2H), 7.57 (d, 1H), 7.65 (d, 1H), 7.79 (s, 1H); ¹³C-NMR (CDCl₃, 150 MHz): δ 3.3, 4.0, 10.6, 53.8, 62.9, 11.3, 120.7, 121.2, 123.3, 125.8, 127.0, 129.1, 129.4, 129.8, 130.5, 133.8, 134.1, 139.7, 157.8; HPLC-MS (ESI-TOF): rₜ=9.86 min m/z=417 [M+H]⁺; HRMS (ESI-TOF) m/z calcd for C₂₁H₁₉BrClNO 415.0339, found 415.0338.

### PB14: 6-chloro-3-(1-(4-chlorobenzyl)-4-phenyl-1H-imidazol-5-yl)-1H-indole-2-carboxylic acid

The title compound was synthesized as illustrated below and described in an article by Popowicz, G. M., et al., Cell Cycle 9: 1104-11 (2010).

### a. Ethyl 6-chloro-1H-indole-2-carboxylate

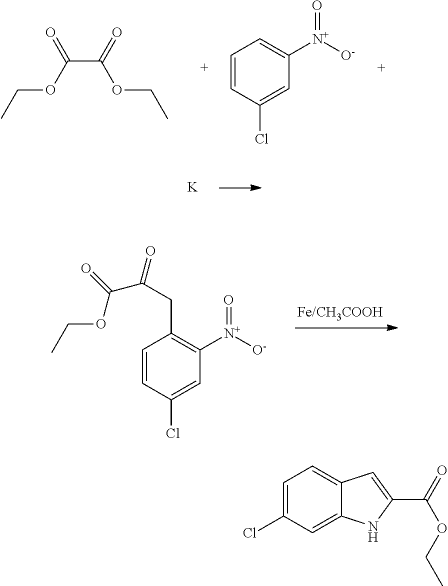

To a mixture of potassium (7.2 g, 185 mmol) in diethyl ether (60 mL) was added an ethanol (40 mL) diethyl ether (100 mL) mixture, followed by the addition of an ether solution of diethyl oxalate (27.8 g, 190 mmol, in diethyl ether, 100 mL). To this reaction mixture was added a solution of 4-chloro-2-nitrotoluene (27.4 g, 160 mmol) in diethyl ether (40 mL). After stirring for 15 h the reaction mixture is sonicated for an additional 7 h. The reaction is stopped by pouring the mixture into 1 N HCl (200 mL) at 0° C. followed by extraction of the aqueous mixture with ethyl acetate. The combined organic layers were washed with brine, dried (anhydrous sodium sulfate) and concentrated to afford the intermediate ethyl 3-(4-chloro-2-nitrophenyl)-2-oxopropanoate which was used directly in the next step. ¹H NMR of the crude product indicates that the conversion is about 80%.

To crude ethyl 3-(4-chloro-2-nitrophenyl)-2-oxopropanoate (ca 130 mmol) in ethanol (260 mL) and glacial acetic acid (260 mL) was added iron powder (74.4 g, 1.33 mol) and the reaction mixture was heated at reflux for 4 h. At the end of the reflux, the mixture was cooled, filtered and the filtrate was evaporated. The obtained residue was partitioned between dichloromethane and 1 N HCl. The organic layer was washed with 1 N HCl, followed by brine and dried (anhydrous sodium sulfate). Evaporation of solvent gave ethyl 6-chloro-1H-indole-2-carboxylate as a pale yellow solid 23 g, (65%) over 2 steps. ¹H NMR (d₆-DMSO, 600 MHz): δ 12.02 (s, 1H), 7.68 (d, J=8.4 Hz, 1H), 7.46 (s, 1H), 7.17 (s, 1H), 7.09 (d, J=8.4 Hz, 1H), 4.34 (t, J=7.2 Hz, 2H), 1.33 (q, J=7.2 Hz, 3H) ppm; ¹³C NMR (d₆-DMSO, 150.92 MHz): δ 161.0, 137.5, 129.2, 128.3, 125.4, 123.7, 120.7, 111.9, 107.8, 60.6, 14.2 ppm. See Lee, K. L., et al., J. Med. Chem., 50: 1380-1400, (2007).

### b. Ethyl 6-chloro-3-formyl-1H-indole-2-carboxylate

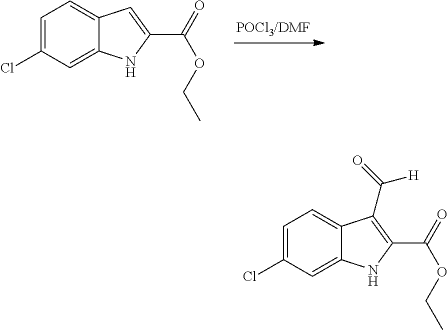

Ethyl 6-chloro-1H-indole-2-carboxylate (4.46 g, 20 mmol), phosphorus oxychloride (3.68 g, 24 mmol) in N,N-dimethyl formamide (15 mL), were added to a 100 mL round bottom flask equipped with stir bar. The reaction was heated to 50° C. for 30 h. After completion, the reaction mixture is quenched with saturated sodium bicarbonate solution (50 mL) and extracted with diethyl ether (3×50 mL). The combined organic phase was washed with brine and dried (anhydrous sodium sulfate). The solvent was evaporated and the crude product was purified by recrystallization (ethyl acetate/hexane) to produce 3.11 g (62%) title compound as light yellow solid. See Di Fabio, R., et. al., Farmaco, 56: 791-798 (2001).

The compound was characterized by ¹H NMR (d₆-DMSO, 600 MHz): δ 12.99 (brs, 1H), 10.63 (s, 1H), 8.26 (d, J=9.0 Hz, 1H), 7.62 (d, J=1.8 Hz, 1H), 7.38 (dd, J=9.0, 1.8 Hz, 1H), 4.51 (q, J=7.2 Hz, 2H), 1.46 (t, J=7.2 Hz, 3H) ppm; ¹³C NMR (d₆-DMSO, 150.92 MHz): δ 187.4, 159.9, 136.1, 133.5, 130.4, 124.0, 123.9, 123.4, 118.2, 112.6 ppm.

### c. Ethyl 6-chloro-3-O-(4-chlorobenzyl)-4-phenyl-1H-imidazol-5-yl)-1H-indole-2-carboxylate

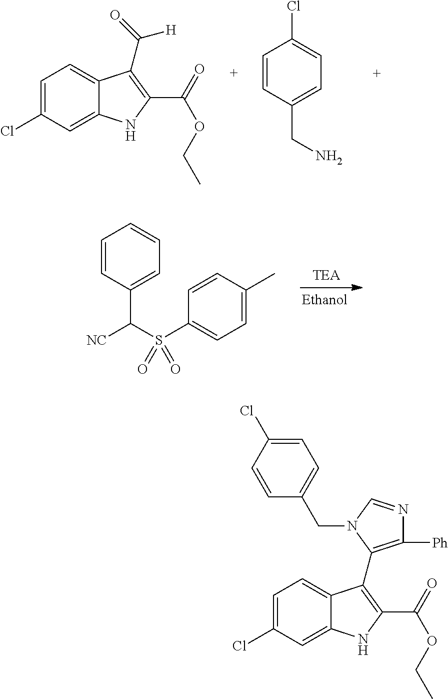

A 20 mL vial equipped with stir bar was charged with ethyl 6-chloro-3-formyl-1H-indole-2-carboxylate (1.06 g, 4.0 mmol), 1-(isocyano(phenyl)methylsulfonyl)-4-methylbenzene (1.10 g, 4.0 mmol), 4-chlorobenzylamine (0.57 g, 4.0 mmol) and triethylamine (0.41 g, 4.0 mmol) in ethanol (10 mL). The reaction was heated at 60° C. for 3 h. After removing the solvent in vaccuo the crude product was purified by silica gel chromatography (0-5% methanol in ethyl acetate) to afford 1.80 g (92%) of the title compound as the light white solid. See Walfrido A. et. al., Bioorg. Med. Chem. Lett., 16: 1740-3, (2006); Domling, A., et. al., ARKIVOC (Gainesville, Fla., United States), 99-109 (2007); Beck B., et. al., QSAR Comb. Chem., 25: 527-535 (2006).

The compound was characterized by ¹H NMR (d₆-DMSO, 600 MHz): δ 12.41 (s, 1H), 8.13 (s, 1H), 7.55 (s, 1H), 7.40 (d, J=7.2 Hz, 2H), 7.12-7.19 (m, 4H), 7.09 (t, J=6.6 Hz, 1H), 7.02 (s, 2H), 6.82 (d, J=8.4 Hz, 2H), 5.00 (s, 2H), 4.08-4.13 (m, 2H), 1.10 (t, J=7.2 Hz, 3H) ppm; ¹³C NMR (d₆-DMSO, 150.92 MHz): δ 160.2, 138.7, 138.3, 136.5, 136.1, 135.0, 131.7, 129.7, 128.7, 128.0, 127.9, 126.8, 125.8, 125.0, 121.9, 121.4, 119.5, 60.4, 47.5, 13.7 ppm; HRMS ESL-TOF for C₂₇H₂₂Cl₂N₃O₂ (M+H⁺) found: m/z: 490.1090; Calc. Mass: 490.1089.

### d. 6-Chloro-3-(1-(4-chlorobenzyl)-4-phenyl-1H-imidazol-5-yl)-1H-indole-2-carboxylic acid (PB14)

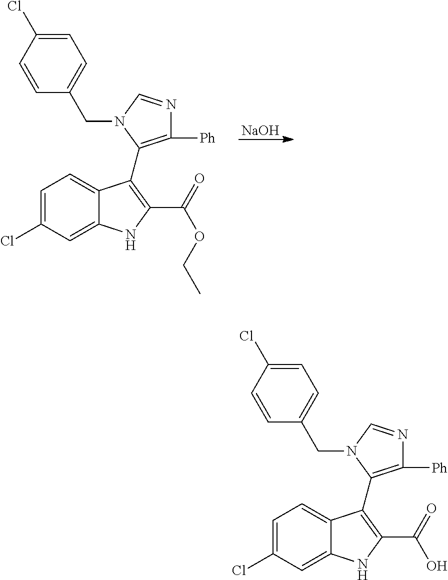

Ethyl 6-chloro-3-(1-(4-chlorobenzyl)-4-phenyl-1H-imidazol-5-yl)-1H-indole-2-carboxylate (900 mg, 2 mmol), NaOH solution (2M, 35 mL) in ethanol (35 mL) were added into a 100 mL round bottom flask equipped with stir bar. The mixture was refluxed for 2.5 h, then poured into a mixture of ice and water. Then 25 mL 4M HCl was added and 3× extracted with ethyl acetate (a 50 mL). The combined organic phase was washed with brine and dried over sodium sulfate. The solvent was removed in vacuum to produce the title compound, 880 mg (95%) as light yellow crystals. ¹H NMR (d₆-DMSO, 600 MHz): δ 12.65 (s, 1H), 9.71 (s, 1H), 7.53 (d, J=1.8 Hz, 1H), 7.40-7.44 (m, 2H), 7.25-7.30 (m, 3H), 7.11 (d, J=8.4 Hz, 2H), 7.02 (d, J=8.4 Hz, 1H), 6.95 (dd, J=8.4, 1.2 Hz, 1H), 6.89 (d, J=8.4 Hz, 2H), 5.27 (d, J=15.0 Hz, 1H), 5.17 (d, J=15.0 Hz, 1H) ppm; ¹³C NMR (d₆-DMSO, 150.92 MHz): δ 161.2, 136.4, 136.2, 133.2, 132.7, 131.2, 129.8, 129.5, 129.3, 129.0, 128.9, 128.1, 127.2, 126.2, 125.5, 122.1, 121.7, 121.3, 49.5 ppm; HRMS ESL-TOF for C₂₅H₁₈Cl₂N₃O₂ (M⁺) found: m/z: 462.0746; Calc. Mass: 462.0776.

### PB15: 4-(6-Chloro-1H-indol-2-yl)-1,3-bis-(4-chlorophenyl)azetidin-2-one

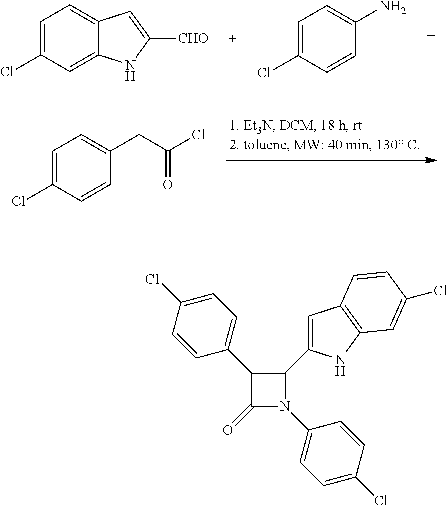

6-Chloro-1H-indole-2-carbaldehyde (90 mg, 0.5 mmol) and 4-chlorophenylamine (65 mg, 0.5 mmol) were dissolved in DCM. A small amount MgSO₄ was added and the mixture was stirred over night. The salt was filtered off and the filtrate was concentrated under reduced pressure. The residue was dissolved in toluene and triethylamine (335 μL, 4.8 eq, 2.4 mmol) and (4-chlorophenyl)acetyl chloride (125.8 μL, 1.72 eq, 0.86 mmol) were added simultaneously. The reaction mixture was heated in the microwave for 40 min at 130° C. After the mixture cooled to room temperature the solid was filtered off and the filtrate evaporated. The residue was dissolved in ethyl acetate and extracted with water and brine. The organic layer was dried over MgSO₄ and evaporated. The crude product was purified by chromatography on silica gel with 4:1 heptane/ethyl acetate to yield 4-(6-chloro-1H-indol-2-yl)-1,3-bis-(4-chlorophenyl)azetidin-2-one (PB13) 54 mg (25%); ¹H-NMR (CDCl₃, 600 MHz): δ 4.58 (s, 1H), 5.12 (s, 1H), 6.61-6.65 (m, 1H), 6.69 (m, 1H), 7.10-7.16 (m, 3H), 7.24-7.32 (m, 4H), 7.37-7.38 (m, 2H), 7.54 (d, 1H), 9.69 (s, 1H); ¹³C-NMR (CDCl₃, 150 MHz): δ 58.6, 62.6, 103.7, 111.4, 116.3, 118.2, 121.2, 121.6, 126.4, 128.6, 128.9, 129.1, 129.4, 129.5, 130.1, 130.2, 132.1, 133.7, 134.4, 135.7, 137.5, 165.7; HPLC-MS (ESI-TOF): rₜ=13.16 min m/z 441 [M+H]⁺; HRMS (ESI-TOF) m/z calcd for C₂₃H₁₅Cl₃N₂O 440.0249, found 440.0254.

Antagonism of the p53-MDM Complex

The inventive compounds bind tightly to MDM a negative regulator of the tumor suppressor p53 protein. Table 3 provides representative binding constants for a diverse family of scaffolds identified as lead compounds from in silico docking studies. It is readily apparent from the illustrated data that the Formula I imidazolines bind more tightly to hMDM2 than many of the other compounds. For example, PB2 and PB11 show low micromolar and submicromolar affinities for hMDM2. Resolution of racemate PB2 into the corresponding syn- and anti-isomers illustrates the influence of chirality in binding, with the syn-isomer being more than an order of magnitude more potent than the anti-isomer. See Table 3. The binding data illustrated in Table 3 was calculated using a variety of analytical techniques, including without limitation, binary titration of an appropriate compound using isotopically enriched ¹⁵N hMDM2 and ¹⁵N-HSQC NMR, antagonist induce dissociation assay (AIDA), NMR-based competitive binding assay between an MDM2 ligand and p53 for hMDM2 protein, surface plasmon resonance studies (SPR) and fluorescent polarization assay.

TABLE 3

| | Binding to Hdm2 | Molecular Weight | Method of K_{D} |
|---|---|---|---|
| Compound | (□M) | [g/mol] | calculation^{[a]} |
| PB1 | 40 ± 15 | 416.74 | bin. titr |
| syn-PB2 | 3 ± 1 | 368.87 | bin. titr., AIDA |
| anti-PB2 | 40 ± 10 | 368.87 | bin. titr. |
| PB3 | 20 ± 7 | 340.45 | bin. titr., AIDA |
| PB4 | 30 ± 10 | 362.25 | bin. titr. |
| PB5 | 30 ± 10 | 337.81 | AIDA |
| PB6 | precipitation | 385.30 | bin. titr. AIDA |
| PB7 | >100 | 379.91 | bin. titr. |
| PB8 | 60 ± 20 | 365.91 | bin. titr. AIDA |
| PB9 | 60 ± 30 | 360.89 | bin. titr. |
| PB10 | 5 ± 0.2 | 507.14 | AIDA, FP |
| PB11 | 0.8 ± 0.4 | 439.02 | bin. titr., AIDA |
| PB12 | 30 ± 11 | 376.27 | bin. titr., AIDA |
| PB13 | precipitation | 416.75 | bin. titr. |
| PB14 | 0.9 ± 0.04 | 462.34 | FP AIDA |
| PB15 | precipitation | 441.74 | bin. titr. |
| Nutlin-3 | 0.09 | 581.5 | SPR |
| MI-63 | 0.03 | 577.5 | FP |

^{[a]}bin. titr, the binary titration of a ligand with the apo-¹⁵N-Hdm2 protein using ¹⁵N-HSQC; AIDA (antagonist induced dissociation assay), the competition NMR experiment between ligand and the p53-Hdm2 complex; FP, fluorescent polarization assay; SPR, surface plasmon resonance.

### Fluorescence Polarization Binding Assays

All fluorescence experiments were performed as described by Czarna, et al., Cell Cycle 8: 1176-84 (2009). Briefly, the fluorescence polarization experiments were read on an Ultra Evolution 384-well plate reader (Tecan) with the 485 nm excitation and 535 nm emission filters. The fluorescence intensities parallel (Intₚₐᵣₐₗₗₑₗ) and perpendicular (Int_{perpedicular}) to the plane of excitation were measured in black 384-well NBS assay plates (Corning) at room temperature (˜20° C.). The background fluorescence intensities of blank samples containing the references buffer were subtracted and steady-state fluorescence polarization was calculated using the equation: P=(Intₚₐᵣₐₗₗₑₗ−GInt_{perpedicular})/(Mtₚₐᵣₐₗₗₑₗ+GInt_{perpedicular}).

A correction factor G (G=0.998 determined empirically) was introduced to eliminate differences in the transmission of vertically and horizontally polarized light. All fluorescence polarization values were expressed in millipolarization units (mP). The binding affinities of the fluorescent p53-derived peptide, see Hu, et al., Cancer Res. 67: 8810-17 (2006), and of the P4 peptide in Czarna, et al. (2009), supra, towards MDM2 and MDMX proteins were determined in buffer containing 50 mM NaCl, 10 mM Tris pH 8.0, 1 mM EDTA, 10% DMSO. Each sample contained 10 nM of the fluorescent P4 peptide and MDM2 (the MDM2 concentration used, from 0 to 1 μM and MDMX, from 0 to 10 μM) in a final volume of 50 μl. Competition binding assays were performed using the 10 nM fluorescent P4 peptide, 15 nM MDM2 or 120 nM MDMX. Plates were read at 30 min after mixing all assay components. Binding constant and inhibition curves were fitted using the SigmaPlot (SPSS Science Software).

### Computational Library Generation and Docking

Used for this study was a database of several hundred scaffolds, amenable by MCR chemistry in one or two synthetic transformations served. Virtual libraries were generated using REACTOR software pursuant to Pirok, et al. (2006), supra. The anchor group, for instance, the indole or a 4-chlorophenyl group, was included in each scaffold at different variable positions. The other positions of each scaffold were complemented by substitutents derived from commercially available starting materials covering a broad physicochemical property space, e.g. aliphatic, aromatic, small, bulky substitutents. All possible stereoisomers to a particular compound were created. Three-dimensional coordinates of the created SMILES libraries was achieved using OMEGA software. See VIRTUAL SCREENING IN DRUG DISCOVERY, J. Alvarez and B. Schoichet, CRC Press (2005), at Sect. 12.3.1. Constrained docking including energy minimization was performed using MOLOC software using the template matching routine. See Gerber, P., J. Comp.-Aided Mol. Des. 12: 37-51 (1998), and loc. cit. 9: 251-68 (1995).

The resulting docking models of the virtual MCR molecules were visually inspected and ranked. A higher rank was assigned to compounds that provided substituent groups capable of occupying the Leu26 and Phe19 pockets, in addition to binding interactions between the anchor group and Trp23 pocket of hMDM2.

What is claimed is:

1. A compound of Formula I 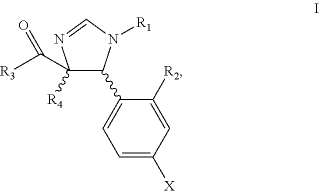
wherein:
R₁ is selected from the group consisting of phenyl, alkyl, aryl, cycloalkyl, cycloalkylalkylene, arylalkylene, heterocycle, and heterocycloalkyl,
wherein when R₁ is phenyl or arylalkylene, R₁ is substituted with one or more substituents selected from the group consisting of (C₁-C₈)alkyl, (C₁-C₈)haloalkyl, —Cl, —Br, —I, —F —NO₂, and (C₁-C₆)hydroxyalkyl;
X is selected from the group consisting of Cl, F, Br and I;
R₂ is selected from the group consisting of —H, —OH, and NR^{a}R^{b},
wherein R^{a} and R^{b} are each independently selected from the group consisting of hydrogen, (C₁-C₈)alkyl, aryl, heteroaryl, heterocycloalkyl, and (C₁-C₆)hydroxyalkyl group;
R₃ is selected from the group consisting of alkoxy, and —NHR^{c} group,
wherein R^{c} is selected from the group consisting of hydrogen, cycloalkylakylene, alkoxy-(C₁-C₈)alkylene, (C₃-C₈)heterocycloalkyl-(C₁-C₈)alkylene, (C₃-C₈)heteroaryl-(C₁-C₆)alkylene, amino-(C₁-C₈)alkylene, hydroxyalkylene, and (W)—(CH₂)ₘ—O—(CH₂)ₙ—,
wherein W is selected from the group consisting of —OH, and NR^{a}R^{b}, and
m and n are each independently an integer in the range from 1 to 8 inclusive; and
R₄ is selected from the group consisting of aryl, phenyl, benzyl, heteroaryl, heteroaryl-(C₁-C₈)alkylene, and aryl-(C₁-C₈)alkylene,
wherein when R₄ is phenyl or benzyl, R₄ is substituted with one or more substituents selected from the group consisting of (C₁-C₈)alkyl, (C₁-C₈)haloalkyl, —Cl, —Br, —I, —F —NO₂, and (C₁-C₆)hydroxyalkyl.

2. The compound of claim 1, wherein X is halogen.

3. The compound of claim 1, wherein R₂ is hydroxy.

4. The compound of claim 1, wherein R₂ is hydroxy and X is halogen.

5. The compound of claim 4, wherein R₂ is hydroxy and X is chlorine.

6. The compound of claim 1, selected from the group consisting of

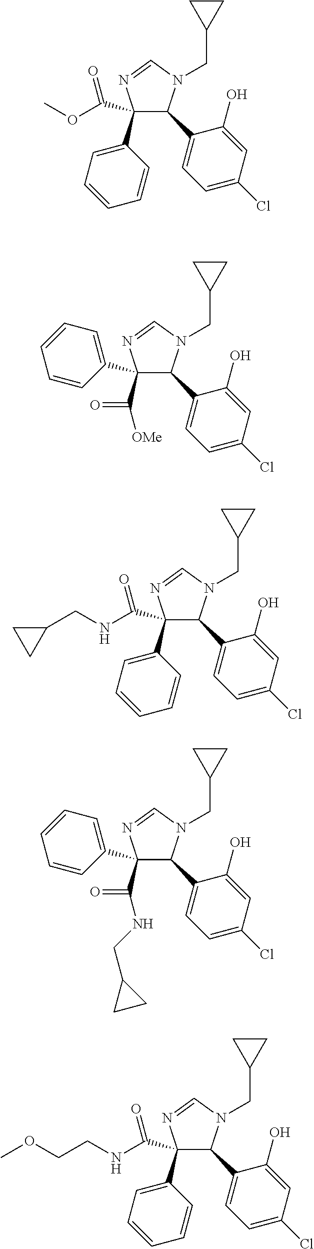

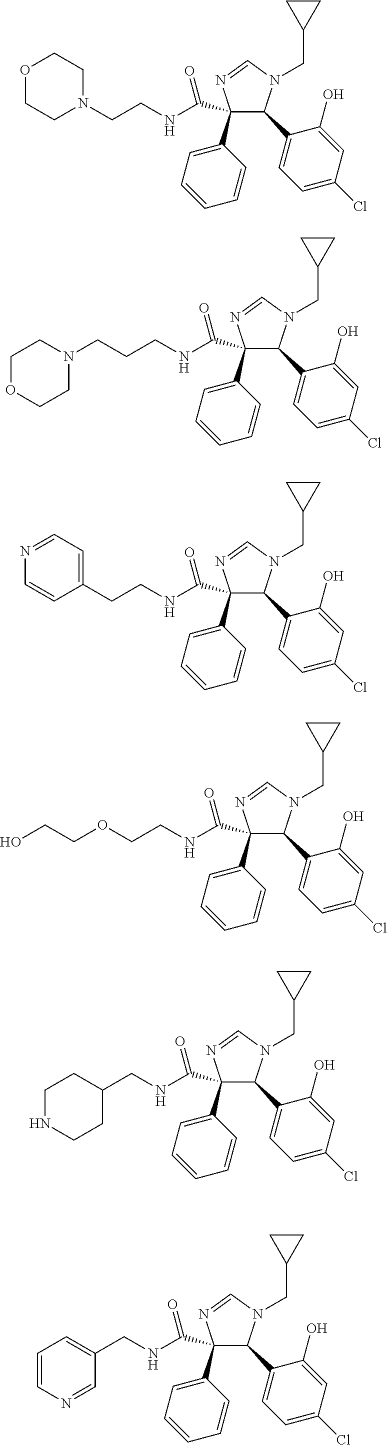

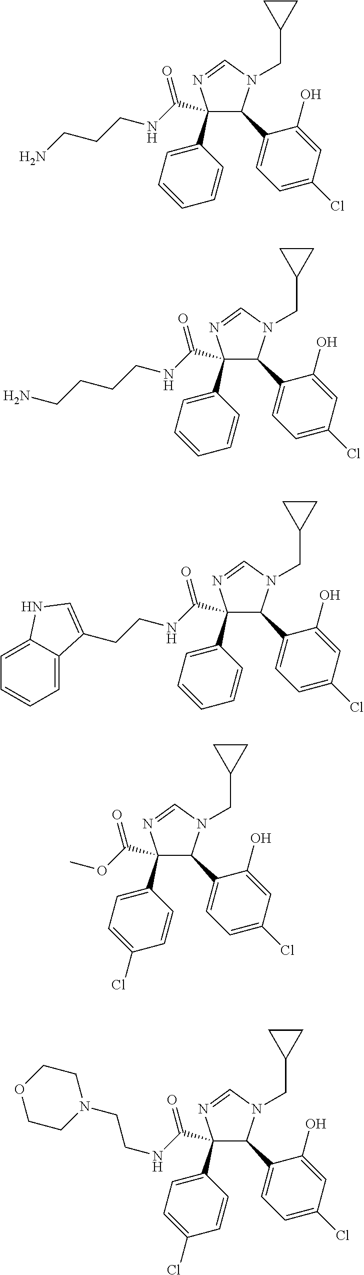

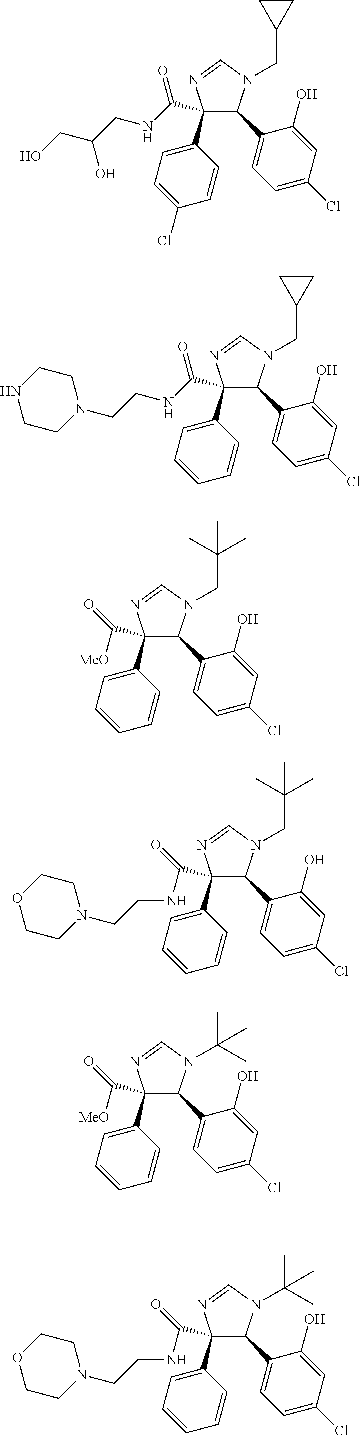

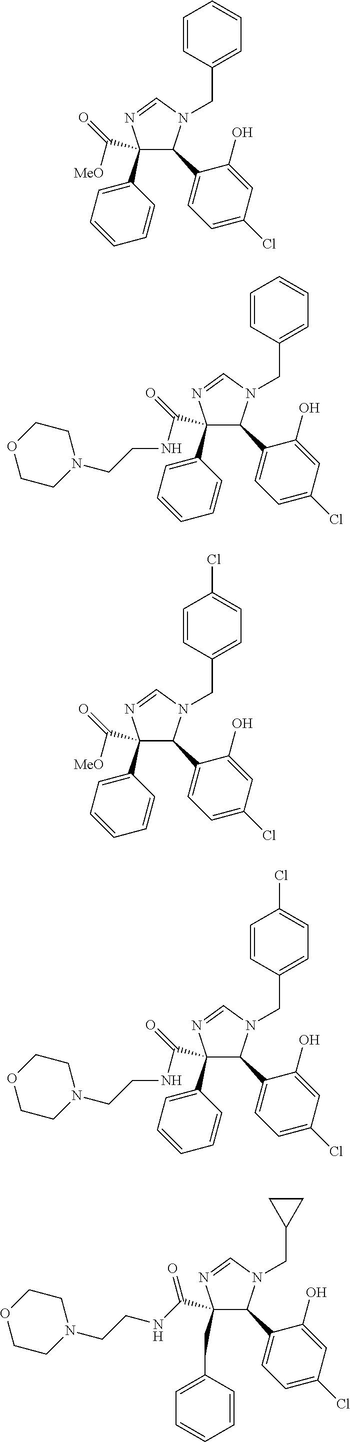

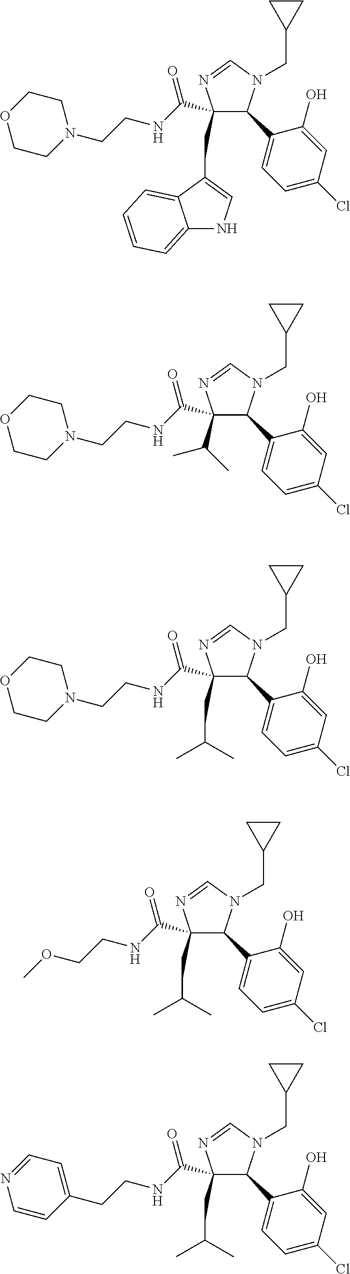

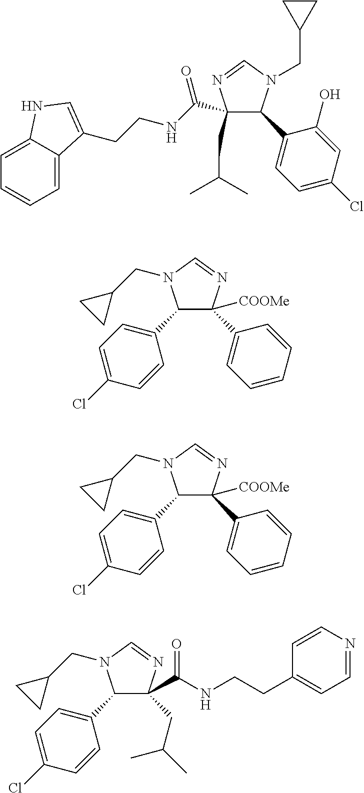

7. The compound according to claim 6, wherein the compound is

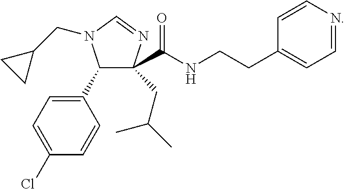

8. A pharmaceutical composition comprising a therapeutically effective amount of at least one compound according to Formula I, 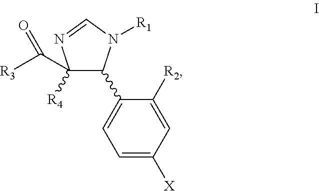
wherein:
R₁ is selected from the group consisting of phenyl, alkyl, aryl, cycloalkyl, cycloalkylalkylene, arylalkylene, heterocycle, and heterocycloalkyl,
wherein when R₁ is phenyl or arylalkylene, R₁ is substituted with one or more substituents selected from the group consisting of (C₁-C₈)alkyl, (C₁-C₈)haloalkyl, —Cl, —Br, —I, —F —NO₂, and (C₁-C₆)hydroxyalkyl;
X is selected from the group consisting of Cl, F, Br and I;
R₂ is selected from the group consisting of —H, —OH, and NR^{a}R^{b},
wherein R^{a} and R^{b} are each independently selected from the group consisting of hydrogen, (C₁-C₈)alkyl, aryl, heteroaryl, heterocycloalkyl, and (C₁-C₆)hydroxyalkyl group;
R₃ is selected from the group consisting of alkoxy, and —NHR^{c} group,
wherein R^{c} is selected from the group consisting of hydrogen, cycloalkylakylene, alkoxy-(C₁-C₈)alkylene, (C₃-C₈)heterocycloalkyl-(C₁-C₈)alkylene, (C₃-C₈)heteroaryl-(C₁-C₆)alkylene, amino-(C₁-C₈)alkylene, hydroxyalkylene, and (W)—(CH₂)ₘ—O—(CH₂)ₙ—,
wherein W is selected from the group consisting of —OH, and NR^{a}R^{b}, and m and n are each independently an integer in the range from 1 to 8 inclusive; and
R₄ is selected from the group consisting of aryl, phenyl, benzyl, heteroaryl, heteroaryl-(C₁-C₈)alkylene, and aryl-(C₁-C₈)alkylene,
wherein when R₄ is phenyl or benzyl, R₄ is substituted with one or more substituents selected from the group consisting of (C₁-C₈)alkyl, (C₁-C₈)haloalkyl, —Cl, —Br, —I, —F —NO₂, and (C₁-C₆)hydroxyalkyl.

9. The pharmaceutical composition according to claim 8, wherein the compound is selected from the following table:

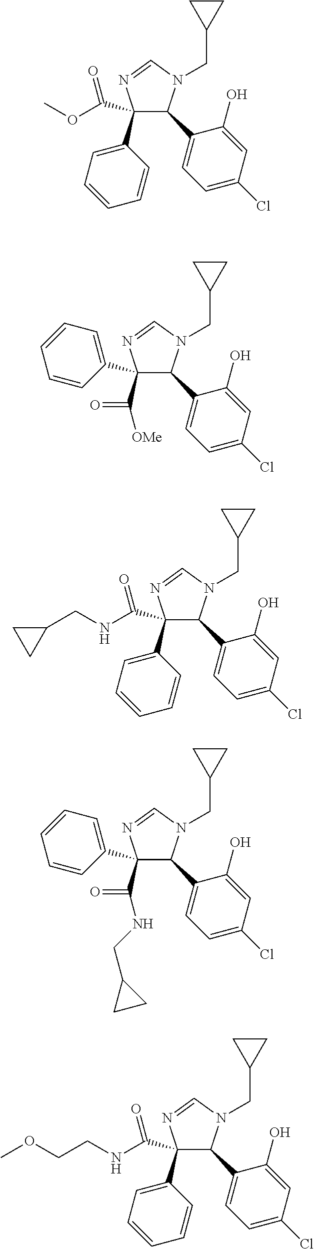

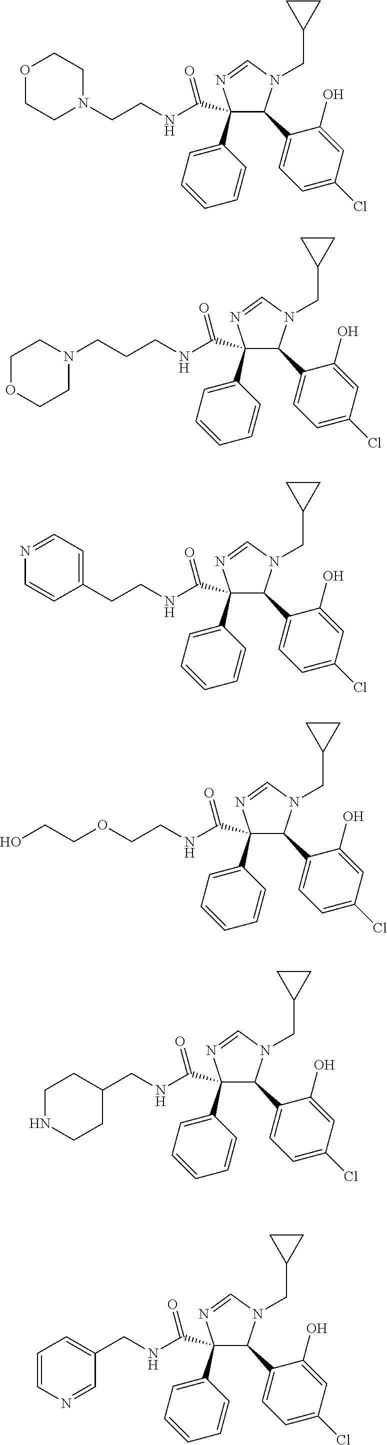

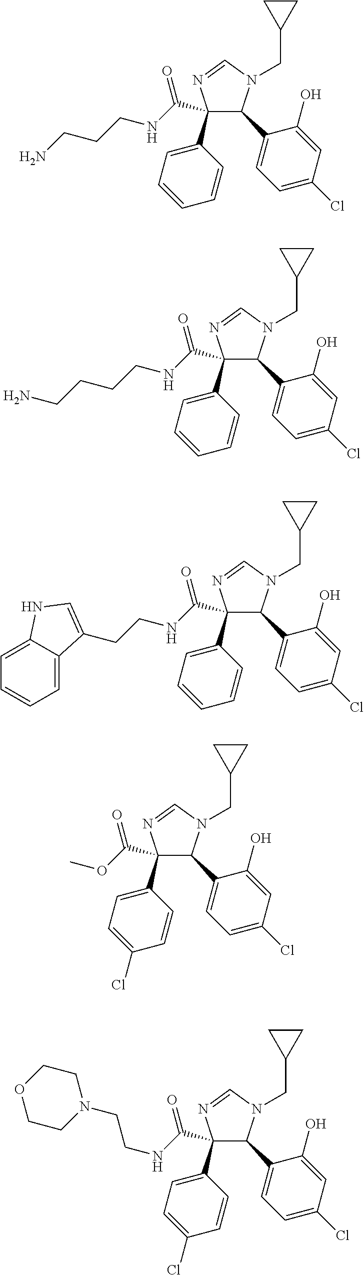

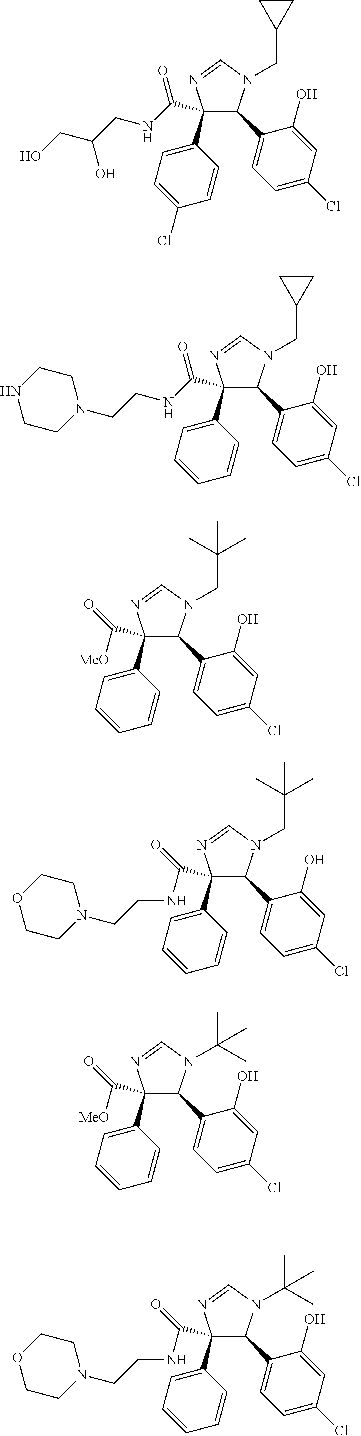

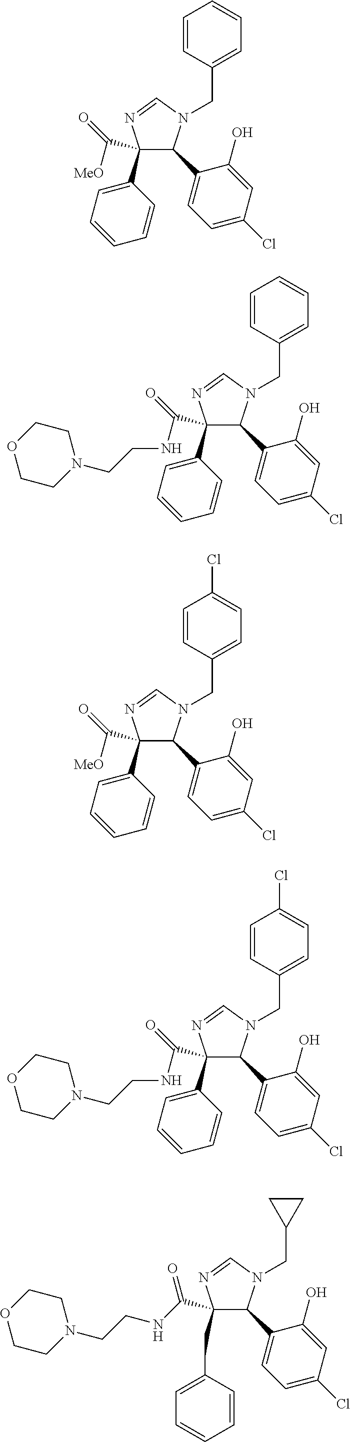

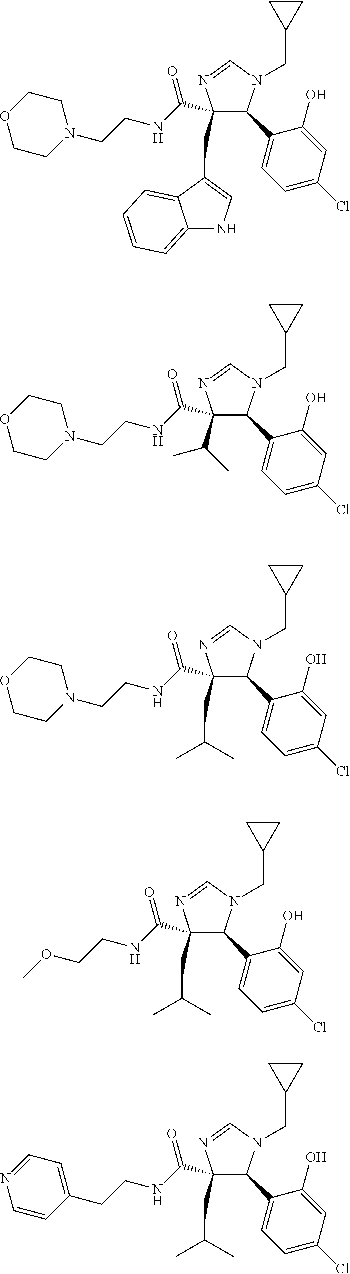

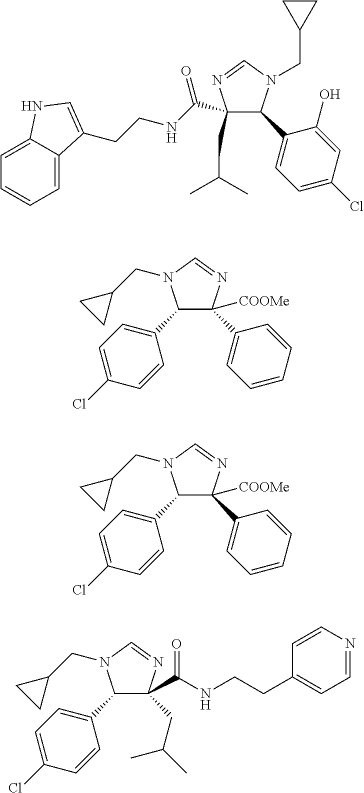

10. The pharmaceutical composition according to claim 9, wherein the compound is [chemical structure]
